SEQUENCE LISTING (10 sequences)

SEQ ID NO: 1 - protein, 1273 aa, coronavirus SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1273 aa, coronavirus SARS-CoV-2
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1273 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 236 aa, Homo sapiens
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 5 - protein, 236 aa, Homo sapiens; identical to SEQ ID NO: 4

SEQ ID NO: 6 - protein, 236 aa, synthetic construct; identical to SEQ ID NO: 4

SEQ ID NO: 7 - protein, 29 aa, synthetic construct
GGGSNWSHPQFEKGGGSNWSHPQFEKGGG

SEQ ID NO: 8 - protein, 25 aa, synthetic construct
SGRTQISSSSFEFCSRRYQSPGPAA

SEQ ID NO: 9 - DNA, 23 bp, synthetic construct
  source 1..23: mol_type = other DNA
cctgctgaggatcaattacactc

SEQ ID NO: 10 - DNA, 19 bp, synthetic construct
  source 1..19: mol_type = other DNA
ggatgcttcccagaaacca

(19) United States
(12) Patent Application Publication
(10) Pub. No.: US 2026/0337249 A1
(43) Pub. Date: Oct. 29, 2026

(54) SYNTHETIC TETRASPANINS AND EXTRACELLULAR VESICLES COMPRISING THE SAME

(71) Applicant: The University of North Carolina at Chapel Hill, Chapel Hill, NC (US)

(72) Inventors: Dirk Dittmer, Chapel Hill, NC (US); Ryan McNamara, Boston, MA (US); Yijun Zhou, Rockville, MD (US); Allaura Cone, Durham, NC (US)

(21) Appl. No.: 19/114,941

(22) PCT Filed: Sep. 26, 2023
(86) PCT No.: PCT/US2023/075101

§ 371 Date: Mar. 25, 2025

Related U.S. Application Data
(60) Provisional application No. 63/377,243, filed on Sep. 27, 2022.

(51) Int. Cl. C07K 14/705; A61K 39/00; A61K 39/215; A61P 37/04; C07K 14/005

(52) U.S. Cl. CPC C07K 14/705; A61K 39/215; A61P 37/04; C07K 14/005; A61K 2039/55555; C07K 2319/03; C07K 2319/60; C12N 2770/20022; C12N 2770/20034

(57) ABSTRACT

This invention relates synthetic tetraspanin proteins comprising a heterologous protein or fragment thereof inserted within the second extracellular loop of a tetraspanin backbone, wherein the heterologous protein or fragment thereof is at least 100 amino acids in length. The invention further relates to nucleic acid molecules encoding the same, vectors, cells, and compositions comprising the same, and methods of using the same.

### STATEMENT OF PRIORITY

This application cliams the benefit, under 35 U.S.C. § 119 (e), of U.S. Provisional Application No. 63/377,243, filed Sep. 27, 2022, the entire contents of which is incorporated by reference herein in its entirety.

### STATEMENT OF GOVERNMENT SUPPORT

This invention was made with government support under Grant Number DE018304, DA040394, CA019014, and CA228172 awarded by the National Institutes of Health. The government has certain rights in the invention.

### STATEMENT REGARDING ELECTRONIC FILING OF A SEQUENCE LISTING

A Sequence Listing in XML format, entitled 5470-935WO_ST26.xml, 14,174 bytes in size, generated on Sep. 26, 2023, and filed herewith, is hereby incorporated by reference in its entirety for its disclosures.

### FIELD OF THE INVENTION

This invention relates synthetic tetraspanin proteins comprising a heterologous protein or fragment thereof inserted within the second extracellular loop of a tetraspanin backbone, wherein the heterologous protein or fragment thereof is at least 100 amino acids in length. The invention further relates to nucleic acid molecules encoding the same, vectors, cells, and compositions comprising the same, and methods of using the same.

### BACKGROUND OF THE INVENTION

Tetraspanins including CD9, CD81, and CD63 are a family of proteins that span the cell membrane four (tetra) times. They are crucial components of extracellular vesicles (EVs) also known as exosomes, and are specifically transported into EVs.

EVs are small membrane-bound particles involved in cell-to-cell communication, and can functionally transfer biological material between cells and tissues without inducing inflammatory or auto-immune cascades. EVs package various cargo, such as proteins, lipids, and nucleic acid. EVs are secreted by all cell types and found in all bodily fluids evaluated (Colombo et al. 2014 Annu Rev Cell Dev Biol 30:255-289). EVs have been studied to treat inflammation and neurodegenerative diseases (Li and Hua 2017 Cell Mol Life Sci 74 (13) 2345-2360; Fu et al. 2019 Cells 8 (8): 784; Cone et al. 2021 Theranostics 11 (17): 8129-8142).

The present invention overcomes previous shortcomings in the art by providing synthetic tetraspanin proteins, and methods of making and using the same.

### SUMMARY OF THE INVENTION

One aspect of the present invention provides a synthetic tetraspanin protein comprising: a tetraspanin backbone comprising an N-terminus, a C-terminus, a first, second, third and fourth transmembrane segment, a first extracellular loop between the first and second transmembrane segment, and a second extracellular loop between the third and fourth transmembrane segments; and a heterologous protein or fragment thereof inserted within the second extracellular loop of the tetraspanin backbone, wherein the heterologous protein or fragment thereof is at least 100 amino acids in length.

Also provided herein are isolated nucleic acid molecules encoding a synthetic tetraspanin protein of the present invention.

Also provided herein are vectors comprising and/or encoding a synthetic tetraspanin protein of the present invention.

In some embodiments, a vector of the present invention may be an extracellular vesicle, including but not limited to an exosome, an ectosome, a microvesicle, a microparticle, an exosome-like vesicle, or any combination thereof.

Also provided herein are populations of vectors (e.g., populations of extracellular vesicles) and compositions comprising a synthetic tetraspanin protein of the present invention.

Also provided herein are cells comprising a synthetic tetraspanin of the present invention (e.g., an immortalized cell, e.g., a cell line).

Another aspect of the present invention provides a method of producing an immune response to an infection in a subject, comprising administering to the subject an effective amount of a synthetic tetraspanin protein, nucleic acid molecule, vector, population, and/or composition of the present invention.

Another aspect of the present invention provides a method of preventing a disorder associated with an infection in a subject, comprising administering to the subject an effective amount of a synthetic tetraspanin protein, nucleic acid molecule, vector, population, and/or composition of the present invention.

Another aspect of the present invention provides a method of protecting a subject from the effects of an infection, comprising administering to the subject an effective amount of a synthetic tetraspanin protein, nucleic acid molecule, vector, population, and/or composition of the present invention.

Another aspect of the present invention provides a method of treating to an infection in a subject, comprising administering to the subject (e.g., the subject having or suspected of having or developing the infection) an effective amount of a synthetic tetraspanin protein, nucleic acid molecule, vector, population, and/or composition of the present invention.

Also provided herein is a method of producing an immune response to a coronavirus infection (e.g., a SARS-COV-2 infection, e.g., COVID-19 and/or a variant thereof) in a subject, comprising administering to the subject an effective amount of a synthetic tetraspanin protein, nucleic acid molecule, vector, population, and/or composition of the present invention.

Also provided herein is a method of preventing a disorder associated with a coronavirus infection (e.g., a SARS-COV-2 infection, e.g., COVID-19 and/or a variant thereof) in a subject, comprising administering to the subject an effective amount of a synthetic tetraspanin protein, nucleic acid molecule, vector, population, and/or composition of the present invention.

Also provided herein is a method of protecting a subject from the effects of a coronavirus infection (e.g., a SARS-COV-2 infection, e.g., COVID-19 and/or a variant thereof), comprising administering to the subject an effective amount of a synthetic tetraspanin protein, nucleic acid molecule, vector, population, and/or composition of the present invention.

Also provided herein is a method of treating to a coronavirus infection (e.g., a SARS-CoV-2 infection, e.g., COVID-19 and/or a variant thereof) in a subject, comprising administering to the subject (e.g., the subject having or suspected of having or developing a coronavirus infection) an effective amount of a synthetic tetraspanin protein, nucleic acid molecule, vector, population, and/or composition of the present invention.

In some embodiments, administering an effective amount of the synthetic tetraspanin protein, nucleic acid molecule, vector, population and/or composition to the subject may comprise administering an effective amount of the synthetic tetraspanin protein, nucleic acid molecule, vector, population and/or composition to the subject prior to the subject developing symptoms of the infection (e.g., comprising administering prophylactically, e.g., as a prophylactic vaccine).

In some embodiments, the synthetic tetraspanin protein may stimulate neutralizing antibodies.

Another aspect of the present invention provides a method of producing an extracellular vesicle comprising a synthetic tetraspanin: delivering a synthetic tetraspanin protein, nucleic acid molecule, vector, population, and/or composition of the present invention, to a cell capable of producing extracellular vesicles (e.g., contacting the cell, e.g., transfecting the cell), thereby producing a cell producing one or more extracellular vesicles comprising the synthetic tetraspanin; and isolating the one or more extracellular vesicles comprising the synthetic tetraspanin.

### BRIEF DESCRIPTION OF THE DRAWINGS

FIG. 1 shows an example generic stepwise schematic illustrating the synthetic tetraspanin of the invention.

FIG. 2 shows a schematic of an example tetraspanin as delivery/expression vector into an EV. FIG. 2 panel A shows the vector on top and two different variants, SARS-COV-2 (strain Wuhan or strain Delta) inserted into the tetraspanin. FIG. 2 panel B shows a schematic of the amino acid positions of the four transmembrane domains, an example Spike domain insertion, and another construct that does not carry the GFP tag. FIG. 2 panel C shows the exact position of various aspects of an example synthetic tetraspanin of the present invention. Strep tag sequence shown is SEQ ID NO:7; linker sequence shown is SEQ ID NO:8. (29) LGVALWLRHD. . . . PQTTNLLYLE (77) refers to amino acid residue positions 29 to 48 of the reference CD81 antigen sequence NCBI Accession No. NP_004347.1 (SEQ ID NO:4), with the 29 amino acid in length strep tag sequence (SEQ ID NO:7) inserted at the indicated ( . . . ) location between residues 38 and 39. (170) NNAKAVVKTF. . . . HETLDCCGSS refers to amino acid residue positions 141 to 160 of SEQ ID NO:4 as the location for insertion of the cargo (e.g., viral Spike glycoprotein) on CD81 (amino acid position numbering shown in figure is adjusted for inclusion of the 29 amino acid in length strep tag sequence (SEQ ID NO:7) as described above). (256) CCGIRNSSVY. . . . MVSKGEELFT (300) refers to amino acid residue positions 227 to 236 of SEQ ID NO:4, where a linker may be inserted at the indicated ( . . . ) location, with amino acid position numbering shown in the figure again adjusted for inclusion of the 29 amino acid in length strep tag sequence (SEQ ID NO:7) and the inclusion of the cargo (e.g., viral glycoprotein) as described above.

FIG. 3 shows the SARS-COV-2 Spike protein expressed and localized in an exosome, correctly folded.

FIG. 4 shows a cryo-electron microscopy image of an exosome expressing the Spike protein.

FIG. 5 shows an immunofluorescence micrograph image of an EV comprising a synthetic tetraspanin of the present invention, stained with anti-SARS-COV-2 Spike antibody and anti-CD81 antibody. Top left staining is EV staining, bottom left is anti-CD81 staining, and bottom right is anti-SARS-COV-2 Spike staining.

FIG. 6 shows a schematic of an example experimental design to study immune reactivity upon immunization with and without adjuvant.

FIG. 7 shows a data plot indicating a robust immune response induced in experimental animals. The graph plots the result of a conformation-specific ELISA assay using SARS-COV-2 Spike. IgG antibody concentration is shown on the vertical axis (ng/ml), and the three groups Exo-SD, Exo-SD plus Alum, and PBS plus Alum are shown on the horizontal axis. Differences are significant at p≤0.001 by non-parametric comparison. N=4 animals per group.

FIG. 8 shows images of Western blot assays using mouse antisera to recognize bacterial expressed SARS-COV-2 Spike. Molecular weight markers are shown on the left in each panel.

FIG. 9 lists particulars of example isolated EVs comprising a synthetic tetraspanin of the present invention.

FIG. 10 shows Western blot analysis of the different constructs.

FIG. 11 shows images from an immunofluorescence assay of the different stable cell lines. Cells were seeded and transfected with the constructs described. 24 hours post-transfection, cells were fixed and stained with a CD81 antibody.

FIG. 12 shows images of blots and data graphs quantifying the characterization of EVs purified from cells. (FIG. 12 panel A) Western blot analysis of EVs from control cell lysate, backbone vector, Exo-WT Spike, and Exo-Delta Spike. (FIG. 12 panel B) Nanoparticle tracking analysis of the different CD81-GFP cell lines. (FIG. 12 panel C) Size analysis from NTA of the EVs purified from cells.

FIG. 13 shows images from electron micrographs of EVs purified from CD81-GFP and Exo-Spike cells. (FIG. 13 panel A) Transmission EM of EVs from CD81-GFP expressing cells. (FIG. 13 panel B) TEM of Exo-Spike EVs. (FIG. 13 panel C) Cryo-EM of EVs from CD81-GFP expressing cells. (FIG. 13 panel D) Cryo-EM of Exo-Spike EVs.

FIG. 14 shows images and related charts of direct stochastic optical reconstruction microscopy (dSTORM) analysis of EVs. (FIG. 14 panel A) Antibody only control well to check for background antibody staining. (FIG. 14 panel B) CD81-GFP EVs stained with CD81-488. (FIG. 14 panel C) Exo-Spike EV stained with Spike-568. (FIG. 14 panel D) Exo-Spike EV stained with Spike-568 and CD81-488. (FIG. 14 panel E) Pie chart of percent of EVs stained with Spike and CD81.

FIG. 15 shows images of dSTORM analysis of EVs binding to ACE2. (FIG. 15 panel A) Antibody only control well. (FIG. 15 panel B) CD81-GFP EV stained with CD81-488. (FIG. 15 panel C) Exo-Spike EV binding to purified ACE2. (FIG. 15 panel D) Exo-Spike EVs stained with CD81-488 and binding to ACE2

FIG. 16 shows images and a related data plot of Germinal centers of mice treated with (FIG. 16 panel A) PBS and (FIG. 16 panel B) Exo-Spike. Top left is DAPI, Top right is B220, and bottom left is BCL6. Bottom right is merge. (FIG. 16 panel C) The number of germinal centers in the different treatment groups.

FIG. 17 shows a schematic of the treatment plan and boost regimen tested, as well as images of blots and related data graphs of experiments performed with injection of Exo-Spike in mice to test for presence of antibodies. FIG. 17 panel A: Illustration of experimental design. FIG. 17 panel B: Western blot assay testing sera for presence of anti-SARS-COV-2 Spike antibodies. FIG. 17 panel C: ELISA assay testing for the presence of SARS-COV-2 trimer-Spike antibodies. FIG. 17 panel D: ELISA testing for the presence of the SARS-COV-2 receptor binding domain (RBD)-specific antibodies.

FIG. 18 shows a schematic of the treatment plan and boost regimen repeat experiments tested, as well as images of blots and related data graphs of experiments performed with injection of Exo-Spike in mice to test for presence of antibodies. FIG. 18 panel A: Illustration of experimental design. FIG. 18 panel B: Western blot assay testing sera for presence of anti-SARS-COV-2 Spike antibodies. FIG. 18 panel C: ELISA assay testing for the presence of SARS-COV-2 trimer-Spike antibodies. FIG. 18 panel D: ELISA testing for the presence of the SARS-COV-2 receptor binding domain (RBD)-specific antibodies.

FIG. 19 shows images of B-cell receptor sequencing analyses to determine B-cell repertoire diversity. Larger dots in each graph show areas of high mutation rates. Results shown from analyses performed on recipient mice vaccinated with: (FIG. 19 panel A) PBS, (FIG. 19 panel B) Exo-Spike, (FIG. 19 panel C) Exo-Spike+Alum, and (FIG. 19 panel D) Control.

FIG. 20 shows data graphs related to safety studies performed to test deleterious effects on ice from injection with Exo-Spike. Mice were weight at the start of injections (FIG. 20 panel A) or 6 days before injections were started (FIG. 20 panel B). No deleterious effects on weight of mice were found.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, +1%, ±0.5%, or even±0.1% of the specified value as well as the specified value. For example, “about X” where X is the measurable value, is meant to include X as well as variations of =10%, ±5%, ±1%, ±0.5%, or even±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

As used herein, the term “nucleic acid” encompasses both RNA and DNA, including cDNA, genomic DNA, synthetic (e.g., chemically synthesized) DNA and chimeras of RNA and DNA. The nucleic acid may be double-stranded or single-stranded. The nucleic acid may be synthesized using nucleotide analogs or derivatives (e.g., inosine or phosphorothioate nucleotides). Such nucleotides can be used, for example, to prepare nucleic acids that have altered base-pairing abilities or increased resistance to nucleases.

The terms “nucleic acid segment,” “nucleotide sequence,” “nucleic acid molecule,” or more generally “segment” will be understood by those in the art as a functional term that includes both genomic DNA sequences, ribosomal RNA sequences, transfer RNA sequences, messenger RNA sequences, small regulatory RNAs, operon sequences and smaller engineered nucleotide sequences that express or may be adapted to express, proteins, polypeptides or peptides. Nucleic acids of the present disclosure may also be synthesized, either completely or in part, by methods known in the art.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul al., et Meth. Enzymol. 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (±1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, the term “polypeptide” encompasses both peptides and proteins (including fusion proteins), unless indicated otherwise.

A “fusion protein” is a polypeptide produced when two heterologous nucleotide sequences or fragments thereof coding for two (or more) different polypeptides not found fused together in nature are fused together in the correct translational reading frame.

A “recombinant” nucleic acid, polynucleotide or nucleotide sequence is one produced by genetic engineering techniques.

A “recombinant” polypeptide is produced from a recombinant nucleic acid, polypeptide or nucleotide sequence.

As used herein, an “isolated” polynucleotide (e.g., an “isolated nucleic acid” or an “isolated nucleotide sequence”) means a polynucleotide at least partially separated from at least some of the other components of the naturally occurring organism or virus, for example, the cell or viral structural components or other polypeptides or nucleic acids commonly found associated with the polynucleotide. Optionally, but not necessarily, the “isolated” polynucleotide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polynucleotide is at least about 1%, 5%, 10%, 20%, 30%, 40%, 50%, 60%, 70%, 80%, 90%, 95% or more pure.

An “isolated” polypeptide means a polypeptide that is at least partially separated from at least some of the other components of the naturally occurring organism or virus, for example, the cell or viral structural components or other polypeptides or nucleic acids commonly found associated with the polypeptide. Optionally, but not necessarily, the “isolated” polypeptide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polypeptide is at least about 1%, 5%, 10%, 20%, 30%, 40%, 50%, 60%, 70%, 80%, 90%, 95% or more pure.

Furthermore, an “isolated” cell is a cell that has been partially or completely separated from other components with which it is normally associated in nature. For example, an isolated cell can be a cell in culture medium and/or a cell in a pharmaceutically acceptable carrier.

As used herein with respect to nucleic acids, the term “fragment” refers to a nucleic acid that is reduced in length relative to a reference nucleic acid and that comprises, consists essentially of and/or consists of a nucleotide sequence of contiguous nucleotides identical or almost identical (e.g., 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. In some embodiments, the nucleic acid fragment comprises, consists essentially of or consists of at least about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive nucleotides. In some embodiments, the nucleic acid fragment comprises, consists essentially of or consists of less than about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive nucleotides.

As used herein with respect to polypeptides, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive amino acids.

As used herein with respect to nucleic acids, the term “functional fragment” or “active fragment” refers to nucleic acid that encodes a functional fragment of a polypeptide.

As used herein with respect to polypeptides, the term “functional fragment” or “active fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide (e.g., the ability to up-or down-regulate gene expression). In some embodiments, the functional fragment actually has a higher level of at least one biological activity of the full-length polypeptide.

As used herein, the term “modified,” as applied to a polynucleotide or polypeptide sequence, refers to a sequence that differs from a wild-type sequence due to one or more deletions, additions, substitutions, or any combination thereof. Modified sequences may also be referred to as “modified variant(s).”

As used herein, the term “antigen” refers to a molecule capable of inducing the production of immunoglobulins (e.g., antibodies). As used herein, the term “immunogen” refers to when a molecule is capable of inducing a multi-faceted humoral and/or cellular-mediated immune response. In some embodiments, an antigen may be referred to as an immunogen, e.g., under conditions when the antigen is capable of inducing a multi-faceted humoral and/or cellular-mediated immune response. A molecule and/or composition (e.g., including but not limited to a nucleic acid, protein, polysaccharide, ribonucleoprotein (RNP), whole bacterium, and/or composition comprising the same) that is capable of antibody may be referred to as “antigenic” and/or that is capable of immune response stimulation may be referred to as “immunogenic,” and can be said to have the ability of antigenicity and/or immunogenicity, respectively. The binding site for an antibody within an antigen and/or immunogen may be referred to as an epitope (e.g., an antigenic epitope).

The term “antibody” or “antibodies” as used herein refers to all types of immunoglobulins, including IgG, IgM, IgA, IgD, and IgE. The antibody can be monoclonal or polyclonal and can be of any species of origin, including, for example, mouse, rat, rabbit, horse, goat, sheep or human, or can be a chimeric or humanized antibody. See, e.g., Walker et al., Molec. Immunol. 26:403-11 (1989).

“Effective amount” as used herein refers to an amount of a vector, nucleic acid molecule, epitope, polypeptide, cell, composition or formulation of the invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation.

The term “immunogenic amount” or “effective immunizing dose,” as used herein, unless otherwise indicated, means an amount or dose sufficient to induce an immune response (which can optionally be a protective response) in the treated subject that is greater than the inherent immunity of non-immunized subjects. An immunogenic amount or effective immunizing dose in any particular context can be routinely determined using methods known in the art.

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

By the terms “treat,” “treating” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder. In representative embodiments, the terms “treat,” “treating” or “treatment of” (and grammatical variations thereof) refer to a reduction in the severity of viremia and/or a delay in the progression of viremia, with or without other signs of clinical disease.

A “treatment effective” amount as used herein is an amount that is sufficient to treat (as defined herein) the subject. Those skilled in the art will appreciate that the therapeutic effects need not be complete or curative, as long as some benefit is provided to the subject.

The terms “prevent,” “preventing” or “prevention of” (and grammatical variations thereof) refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. In representative embodiments, the terms “prevent,” “preventing” or “prevention of” (and grammatical variations thereof) refer to prevention and/or delay of the onset and/or progression of viremia in the subject, with or without other signs of clinical disease. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

The efficacy of treating and/or preventing a disorder by the methods of the present invention can be determined by detecting a clinical improvement as indicated by a change in the subject's symptoms and/or clinical parameters (e.g., viremia for a viral infection, etc.), as would be well known to one of skill in the art.

Unless indicated otherwise, the terms “protect,” “protecting,” “protection” and “protective” (and grammatical variations thereof) encompass both methods of preventing and treating a disorder in a subject.

The terms “protective” immune response or “protective” immunity as used herein indicates that the immune response confers some benefit to the subject in that it prevents or reduces the incidence and/or severity and/or duration of disease or any other manifestation of infection. For example, in representative embodiments, a protective immune response or protective immunity results in reduced viremia, whether or not accompanied by clinical disease. Alternatively, a protective immune response or protective immunity may be useful in the therapeutic treatment of existing disease.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

Exosomes are the smallest EV subgroup, ranging in size from 40-150 nanometers (nm). Their surfaces are marked with tetraspanin proteins such as CD9, CD63, and CD81, and internalized proteins comprise many members of the endosomal sorting complex required for transport (ESCRT) machinery (Cocozza et al. 2020 Cell 182 (1): 262; Thery et al. 2018 J Extracell Vesicles 7 (1): 1535750). While not wishing to be bound to theory, EVs are proposed to originate from the inward budding of endosomes into the multivesicular body (MVB) to create intraluminal vesicles (ILVs), which then traffic to the plasma membrane to release the exosomes as a cluster (Pegtel and Gould, 2019 Annu Rev Biochem 88:487-514).

Tetraspanin proteins are signaling proteins enriched in EVs and can serve as an alternate route of exosome biogenesis through an ESCRT-independent pathway. Tetraspanins are four-pass transmembrane proteins on various cell membranes, including the plasma and MVB membranes (Andreu, Z M, 2014 Front Immunol 5:442). Tetraspanins interact with lipids, such as ceramide, or accessory proteins, such as ALIX and Synteinin-1, to create EVs and package specific proteins. For example, while not wishing to be bound to theory, it is proposed that the ESCRT-dependent pathway packages ubiquitinated proteins through Hrs, while CD63 loads non-ubiquitinated proteins into ILVs (Cone et al. 2020 BMC Mol Cell Biol. 21 (1): 58).

Most tetraspanins are well-conserved evolutionarily. Tetraspanins also accumulate in tetraspanin-enriched microdomains (TErM). These TErM domains are essential for exosome biogenesis, protein sorting, antigen presentation, and signaling (Yáñez-Mo et al. Trends Cell Biol. 2009 19 (9): 434-446). One tetraspanin, CD81, is mainly recognized as the hepatitis-C virus receptor (HCV) receptor, but is also crucial for the localization of CD19 and other B-cell receptors (BCR) complexes in tetraspanin-enriched microdomains and presenting MHC class II compartments (Charrin et al. 2014 J Cell Sci 127:3641-3648).

Several evolutionarily distinct viruses usurp exosomes and other EVs. Notable examples include the envelopment of picornaviruses, the transfer of non-coding viral RNAs and proteins during latency of retroviruses and herpesviruses, and the transfer of miRNA by EVs. This can occur both locally and systemically. While not wishing to be bound to theory, much of this virus-EV interplay is believed to go undetected by host immune sensors, such as immunoglobulins or leukocyte antigen recognition/activation, as the inert properties of exosomes and EVs make them a prime target for the transfer of pathogenic material, provided it is internalized and not exposed.

COVID-19 is a positive-sense single-stranded RNA virus with a helically symmetric nucleocapsid and is enveloped. Vaccines against COVID-19 target the virus-encoded Spike protein (Ndwandwe and Wiysonge 2021 Curr Opin Immunol 71:111-116; Byrne and Mclellan 2022 Curr Opin Immunol 77:102209), which binds to the human angiotensin-converting enzyme (ACE2) in the upper and lower respiratory tract. The four primary vaccines against SARS-COV-2 are the two mRNA vaccines, mRNA-1273 (Jackson et al. 2020 N Engl J Med 383 (20:1920-1931) and BNT162b2 (Vogel et al. 2021 Nature 592 (7853): 283-289), a viral vector vaccine Ad26.COV2.S (Sadoff et al., 2021 N Engl J Med 284 (23): 2187-2201), and a protein-based adjuvant vaccine NVX-CoV2373, which is in a clinical trial (Dunkle et al. 2022 N Engl J Med 386 (6): 531-543), the disclosures of each of which are incorporated herein by reference.

The coronavirus Spike protein exists as a homo-trimer, with each unit being extensively glycosylated and having a molecular mass of >180 kDa (Huang et al. 2020 Acta Pharmacol Sin 41 (9): 1141-1149). The intact SARS-COV-2 particle is estimated to contain about 20-40 Spike homotrimers on the surface, giving the virus its crownlike structure when viewed under electron microscopy. The Spike protein of human coronavirus is well-conserved, and studies showing that there is cross reactivation of vaccines to newer variants have been conducted.

The present invention is based, in part, on the discovery that one could establish a platform for inducing immune responses using exosomes as a delivery vehicle for SARS-COV-2 Spike. It was discovered that while the exosomes are immunologically inert, Spike presented on an EV while properly folded could elicit a robust immune activation.

Accordingly, one aspect of the present invention provides a synthetic tetraspanin protein comprising: a tetraspanin backbone comprising an N-terminus, a C-terminus, a first, second, third and fourth transmembrane segment, a first extracellular loop between the first and second transmembrane segment, and a second extracellular loop between the third and fourth transmembrane segments; and a heterologous protein or fragment thereof inserted within the second extracellular loop of the tetraspanin backbone, wherein the heterologous protein or fragment thereof is at least 100 amino acids in length.

In some embodiments, the heterologous protein or fragment thereof may be about 100 amino acids to about 1500 amino acids, e.g., about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, or about 1500 amino acids in length, or any value or range therein. For example, in some embodiments, the heterologous protein or fragment thereof may be about 100 to about 1500, about 500 to about 1000, about 125 to about 1275, about 100 to about 1273, or about 250 to about 1400 amino acids in length. In some embodiments, the heterologous protein or fragment thereof may be at least about 100, 1500, 500, 1000, 125, 1275, 100, 1200, 1273, 250, or at least about 1400 amino acids in length.

In some embodiments, the inserted heterologous protein or fragment thereof may stimulate neutralizing antibodies, e.g., as determined by standard ELISA assay, e.g., pseudo-virus neutralization assay, e.g., ELISA that specifically test for the recognition of non-linear, non-contiguous epitopes.

In some embodiments, the heterologous protein or fragment thereof may express proper protein folding, e.g., may be properly folded. In some embodiments, proper protein folding may comprise native protein secondary, tertiary, and/or quaternary protein folding, and/or proper domain multimerization. Proper folding may be determined by any standard method of the art, such as would be known by the skilled artisan, including but not limited to structural determination assays such as electron microscopy or other functional assays. In some embodiments, proper folding may be determined e.g., by structural determination (e.g., electron microscopy) and/or functional assays, such as recognition of known natural ligands and/or small molecule ligands.

In some embodiments, the heterologous protein or fragment thereof may comprise a viral transmembrane protein or fragment thereof. For example, in some embodiments, the heterologous protein or fragment thereof may comprise a transmembrane protein or fragment thereof of a DNA or RNA virus, such as but not limited to a herpesvirus (e.g. Epstein Barr Virus, e.g., Kaposi Sarcoma associated herpesvirus), a hepatitis virus (e.g., hepatitis A, hepatitis B, and/or hepatitis C), a filovirus (e.g., Ebola virus, e.g., Marburg virus), respiratory syncytial virus (RSV), and a poxvirus (e.g. monkey pox virus). In some embodiments, the heterologous protein or fragment thereof may comprise a coronavirus Spike(S) protein and/or Spike-receptor binding protein (RBD), or any combination thereof. Non-limiting examples of a coronavirus relevant to this invention include e.g., SARS-COV-2, SARS-COV-1, MERS, seasonal coronaviruses (e.g., the common cold), and any variants thereof (e.g., including but not limited to evolving and/or circulating strains thereof). In some embodiments, the heterologous protein or fragment thereof may comprise an HIV gp120 and/or gp41, any fragment and/or variant thereof, or any combination thereof. In some embodiments, a Spike protein of the present invention may include, but is not limited, to any Spike protein or variant thereof as described in EP patent application Ser. No. 17/800,655.7, U.S. patent application Ser. No. 16/344,774, and/or U.S. Provisional Patent Application 62/972,886, the disclosures of each of which are incorporated herein by reference.

In some embodiments, a coronavirus S protein of the present invention may comprise, consist essentially of, or consist of an amino acid sequence at least 70% identical (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical) to:

Spike (Lineage B.1.2., MT565498.1); SEQ ID NO: 1:
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGENCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQGVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT.

In some embodiments, a coronavirus S protein of the present invention may comprise, consist essentially of, or consist of an amino acid sequence at least 70% identical (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical) to:

Delta Spike Variant (SEQ ID NO: 2):
MFVFLVLLPLVSSQCVNLRTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLDVYYHKNNK
SWMESEFGVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YRYRLFRKSNLKPFERDISTEIYQAGSKPCNGVEGENCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQGVNCTEVPVAIHADQLTPTWRVYSTGSNVFQTRAGCL
IGAEHVNNSYECDIPIGAGICASYQTQTNSRRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRENGIGVTQNVLYENQKLIANQFNSAIGKIQDSLSSTASALGKLQN
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT.

In some embodiments, a coronavirus S protein of the present invention may comprise, consist essentially of, or consist of an amino acid sequence at least 70% identical (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical) to:

Stabilized Delta Spike Variant (SEQ ID NO: 3):
MFVFLVLLPLVSSQCVNLRTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLDVYYHKNNK
SWMESEFGVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YRYRLFRKSNLKPFERDISTEIYQAGSKPCNGVEGENCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQGVNCTEVPVAIHADQLTPTWRVYSTGSNVFQTRAGCL
IGAEHVNNSYECDIPIGAGICASYQTQTNSRGSASSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSPIEDLLFNKVTLADAGFIKQYGDCLGDIAARDLI
CAQKFNGLTVLPPLLTDEMIAQYTSALLAGTITSGWTFGAGPALQIPFPM
QMAYRFNGIGVTQNVLYENQKLIANQFNSAIGKIQDSLSSTPSALGKLQN
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT.

In some embodiments, the heterologous protein or fragment thereof may be inserted into the tetraspanin backbone at a position within about amino acid position 114 to about amino acid position 199, wherein the numbering corresponds to the amino acid sequence of a reference tetraspanin identified as GenBank Acc. No. NP_004347.1 and/or CCDS7734.1. GenBank Accession No. NP_004347.1 CD81:

NP_004347.1 CD81 antigen isoform 1
[Homo sapiens]:
SEQ ID NO: 4
MGVEGCTKCIKYLLFVFNFVFWLAGGVILGVALWLRHDPQTTNLLYLELG
DKPAPNTFYVGIYILIAVGAVMMFVGFLGCYGAIQESQCLLGTFFTCLVI
LFACEVAAGIWGFVNKDQIAKDVKQFYDQALQQAVVDDDANNAKAVVKTF
HETLDCCGSSTLTALTTSVLKNNLCPSGSNIISNLFKEDCHQKIDDLFSG
KLYLIGIAAIVVAVIMIFEMILSMVLCCGIRNSSVY.
NCBI Consensus CDS protein set
database (CCDS) 7734.1:
SEQ ID NO: 5
MGVEGCTKCIKYLLFVFNFVFWLAGGVILGVALWLRHDPQTTNLLYLELG
DKPAPNTFYVGIYILIAVGAVMMFVGFLGCYGAIQESQCLLGTFFTCLVI
LFACEVAAGIWGFVNKDQIAKDVKQFYDQALQQAVVDDDANNAKAVVKTF
HETLDCCGSSTLTALTTSVLKNNLCPSGSNIISNLFKEDCHQKIDDLFSG
KLYLIGIAAIVVAVIMIFEMILSMVLCCGIRNSSVY.

In some embodiments, the heterologous protein or fragment thereof may inserted into the tetraspanin backbone immediately following amino acid position 150 (e.g., between amino acid position 150 and 151), wherein the numbering corresponds to the amino acid sequence of the reference tetraspanin identified as GenBank Acc. No. NP_004347.1 and/or CCDS7734.1.

The tetraspanin backbone of the present invention may be any natural, modified, and/or synthetic tetraspanin protein known or later discovered. Non-limiting examples of tetraspanins include any such shown in Table 1. For example, in some embodiments, the tetraspanin backbone may comprise a backbone of any one of Table 1.

In some embodiments, the tetraspanin backbone may comprise, consist essentially of, or consist of an amino acid sequence at least 70% identical (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical) to:

(SEQ ID NO: 6)
MGVEGCTKCIKYLLFVFNFVFWLAGGVILGVALWLRHDPQTTNLLYLELG
DKPAPNTFYVGIYILIAVGAVMMFVGFLGCYGAIQESQCLLGTFFTCLVI
LFACEVAAGIWGFVNKDQIAKDVKQFYDQALQQAVVDDDANNAKAVVKTF
HETLDCCGSSTLTALTTSVLKNNLCPSGSNIISNLFKEDCHQKIDDLFSG
KLYLIGIAAIVVAVIMIFEMILSMVLCCGIRNSSVY

In some embodiments, a synthetic tetraspanin protein of the present invention may further comprise a second insertion within the first extracellular loop. In some embodiments, the second insertion may comprise a detection and/or selection moiety (e.g., a streptavidin tag).

In some embodiments, the second insertion is inserted into the tetraspanin backbone at a position within about amino acid position 35 to about amino acid position 57, wherein the numbering corresponds to the amino acid sequence of a reference tetraspanin identified as GenBank Acc. No. NP_004347.1 and/or CCDS 7734.1. For example, in some embodiments, the second insertion is inserted into the tetraspanin backbone at about position 35, 36, 37, 38, 39, 40, 41, 42, 43, 44, 45, 46, 47, 48, 49, 50, 51, 52, 53, 54, 55, 56, or 57. In some embodiments, the second insertion is inserted into the tetraspanin backbone between amino acid position 38 and 39, e.g., immediately following amino acid position 38.

In some embodiments, the second insertion may comprise, consist essentially of, or consist of an amino acid sequence at least 70% identical (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical) to:

(Streptavidin tag; SEQ ID NO: 7)
GGGSNWSHPQFEKGGGSNWSHPQFEKGGG

In some embodiments, a synthetic tetraspanin protein of the present invention may further comprise a moiety (e.g., a detection and/or selection moiety, e.g., a fluorescence moiety, e.g., green fluorescent protein GFP and/or a derivative thereof, e.g., enhanced green fluorescent protein (EGFP)) at the C-terminus.

In some embodiments, the C-terminal moiety may be attached to the C-terminus via a linker. In some embodiments, the linker may comprise, consist essentially of, or consist of an amino acid sequence at least 70% identical (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical) to:

(linker sequence; SEQ ID NO: 8)
SGRTQISSSSFEFCSRRYQSPGPAA.

The present invention further provides an isolated nucleic acid molecule encoding a synthetic tetraspanin protein of the present invention. In some embodiments, a nucleic acid molecule of this invention may be a cDNA molecule. In some embodiments, a nucleic acid molecule of this invention may be an mRNA molecule.

Also provided is a vector, plasmid or other nucleic acid construct (e.g., a virus vector, e.g., a virus-like particle, a plasmid (e.g., pcDNA3.1 (+) plasmid) e.g., a vesicle, e.g., an extracellular vesicle) comprising and/or encoding the isolated nucleic acid molecule and/or synthetic tetraspanin protein of this invention.

A vector can be any suitable means for delivering a polynucleotide and/or polypeptide to a cell. A vector of this invention can be an expression vector that contains all of the genetic components required for expression of a nucleic acid in cells into which the vector has been introduced, as are well known in the art. The expression vector can be a commercial expression vector or it can be constructed in the laboratory according to standard molecular biology protocols. The expression vector can comprise viral nucleic acid including, but not limited to, poxvirus, vaccinia virus, adenovirus, retrovirus, alphavirus and/or adeno-associated virus nucleic acid. The nucleic acid molecule or vector of this invention can also be in a liposome or a delivery vehicle, which can be taken up by a cell via receptor-mediated or other type of endocytosis. The nucleic acid molecule of this invention can be in a cell, which can be a cell expressing the nucleic acid whereby a synthetic tetraspanin protein of this invention is produced in the cell (e.g., a host cell). In addition, the vector of this invention can be in a cell, which can be a cell expressing the nucleic acid of the vector whereby a synthetic tetraspanin protein of this invention is produced in the cell. It is also contemplated that the nucleic acid molecules and/or vectors of this invention can be present in a host organism (e.g., a transgenic organism), which expresses the nucleic acids of this invention and produces a synthetic tetraspanin protein of this invention. In some embodiments, the vector is a plasmid, a viral vector, a bacterial vector, an expression cassette, a transformed cell, a vesicle (e.g., extracellular vesicle) or a nanoparticle. For example, in some embodiments a synthetic tetraspanin protein of the present invention may be used in combination (e.g., in scaffold(s) and/or conjugated with) other molecules such as, but not limited to, nanoparticles, e.g., as delivery devices.

Types of nanoparticles of this invention for use as a vector and/or delivery device include, but are not limited to, polymer nanoparticles such as PLGA-based, PLA-based, polysaccharide-based (dextran, cyclodextrin, chitosan, heparin), dendrimer, hydrogel; lipid-based nanoparticles such as lipid nanoparticles, lipid hybrid nanoparticles, liposomes, micelles; inorganics-based nanoparticles such as superparamagnetic iron oxide nanoparticles, metal nanoparticles, platin nanoparticles, calcium phosphate nanoparticles, quantum dots; carbon-based nanoparticles such as fullerenes, carbon nanotubes; and protein-based complexes with nanoscales. Types of microparticles of this invention include but are not limited to particles with sizes at micrometer scale that are polymer microparticles including but not limited to, PLGA-based, PLA-based, polysaccharide-based (dextran, cyclodextrin, chitosan, heparin), dendrimer, hydrogel; lipid-based microparticles such as lipid microparticles, micelles; inorganics-based microparticles such as superparamagnetic iron oxide microparticles, platin microparticles and the like as are known in the art. These particles may be generated and/or have materials be absorbed, encapsulated, or chemically bound through known mechanisms in the art.

In some embodiments, the vector is an extracellular vesicle (EV; e.g., an exosome, an ectosome, a microvesicle, a microparticle, an exosome-like vesicle. As used herein, the term “extracellular vesicle” or “EV” refers to a vesicle that is continuously released from a healthy cell (i.e., not from dying cells), independent of the particular biogenesis pathway. EVs are further described in Colombo et al. 2014 Annu Rev Cell Dev Biol 30:255-289; Cocozza et al. 2020 Cell 182 (1): 262; Thery et al. 2018 J Extracell Vesicles 7 (1): 1535750; and Pegtel and Gould, 2019 Annu Rev Biochem 88:487-514, the disclosures of each of which are incorporated herein by reference. In some embodiments, the extracellular vesicle may be an exosome.

Also provided herein is a population of extracellular vesicles comprising a synthetic tetraspanin protein of the present invention.

Also provided in the present invention is a composition comprising a synthetic tetraspanin protein, nucleic acid molecule, vector, and/or population of the present invention. In some embodiments, a composition of the present invention may further comprise a pharmaceutically acceptable carrier, diluent, and/or adjuvant. In some embodiments, the composition may be devoid of adjuvant, pharmaceutically acceptable carrier, and/or diluent.

Also provided herein is an isolated cell (e.g., an immortalized cell, e.g., a cell line) comprising a synthetic tetraspanin, nucleic acid molecule, vector, population and/or composition of the present invention.

By “pharmaceutically acceptable” it is meant a material that is not toxic or otherwise undesirable, i.e., the material may be administered to a subject without causing any undesirable biological effects. For injection, the carrier will typically be a liquid. For other methods of administration (e.g., such as, but not limited to, administration to the mucous membranes of a subject (e.g., via intranasal administration, buccal administration and/or inhalation)), the carrier may be either solid or liquid. For inhalation administration, the carrier will be respirable, and will preferably be in solid or liquid particulate form. The formulations may be conveniently prepared in unit dosage form and may be prepared by any of the methods well known in the art. In some embodiments, that pharmaceutically acceptable carrier can be a sterile solution or composition.

In some embodiments, the present invention provides a pharmaceutical composition comprising a synthetic tetraspanin protein, nucleic acid molecule (e.g., an mRNA molecule), vector, cell, and/or composition of the present invention, a pharmaceutically acceptable carrier, and, optionally, other medicinal agents, therapeutic agents, pharmaceutical agents, stabilizing agents, buffers, carriers, adjuvants, diluents, etc., which can be included in the composition singly or in any combination and/or ratio.

Immunogenic compositions comprising a synthetic tetraspanin protein, nucleic acid molecule (e.g., an mRNA molecule), vector, cell, and/or composition of the present invention may be formulated by any means known in the art. Such compositions, especially vaccines and/or therapeutics, are typically prepared as injectables, either as liquid solutions or suspensions. Solid forms suitable for solution in, or suspension in, liquid prior to injection may also be prepared. Lyophilized preparations are also suitable. In some embodiments, a pharmaceutical composition of the present invention may be a vaccine formulation, e.g., may comprise a synthetic tetraspanin protein, nucleic acid molecule (e.g., an mRNA molecule), vector, cell, and/or composition of the present invention and adjuvant(s), optionally in a vaccine diluent. The active immunogenic ingredients are often mixed with excipients and/or carriers that are pharmaceutically acceptable and/or compatible with the active ingredient. Suitable excipients include but are not limited to sterile water, saline, dextrose, glycerol, ethanol, or the like and combinations thereof, as well as stabilizers, e.g., HSA or other suitable proteins and reducing sugars. In addition, if desired, the vaccines or immunogenic compositions may contain minor amounts of auxiliary substances such as wetting and/or emulsifying agents, pH buffering agents, and/or adjuvants that enhance the effectiveness of the vaccine or immunogenic composition. In some embodiments, a composition of the present invention may be formulated, e.g., as a vaccine, such as for example but not limited to the methods described in PCT/US2017/32287, the disclosures of which are incorporated herein by reference.

In some embodiments, a pharmaceutical composition comprising a synthetic tetraspanin protein, nucleic acid molecule, vector, cell, and/or composition of the present invention may further comprise additional agents, such as, but not limited to, additional antigen as part of a cocktail in a vaccine, e.g., a multi-component vaccine wherein the vaccine may additionally include peptides, cells, virus, viral peptides, inactivated virus, etc. In some embodiments, a pharmaceutical composition comprising a synthetic tetraspanin protein, nucleic acid molecule (e.g., an mRNA molecule), vector, cell, and/or composition of the present invention, and a pharmaceutically acceptable carrier may further comprise an adjuvant. As used herein, “suitable adjuvant” describes an adjuvant capable of being combined with a synthetic tetraspanin protein, nucleic acid molecule (e.g., an mRNA molecule), vector, cell, and/or composition of the present invention to further enhance an immune response without deleterious effect on the subject or the cell of the subject.

The adjuvants of the present invention can be in the form of an amino acid sequence, and/or in the form or a nucleic acid encoding an adjuvant. When in the form of a nucleic acid, the adjuvant can be a component of a nucleic acid encoding the polypeptide(s) or fragment(s) or epitope(s) and/or a separate component of the composition comprising the nucleic acid encoding the polypeptide(s) or fragment(s) or epitope(s) of the invention. According to the present invention, the adjuvant can also be an amino acid sequence that is a peptide, a protein fragment or a whole protein that functions as an adjuvant, and/or the adjuvant can be a nucleic acid encoding a peptide, protein fragment or whole protein that functions as an adjuvant. As used herein, “adjuvant” describes a substance, which can be any immunomodulating substance capable of being combined with a composition of the invention to enhance, improve, or otherwise modulate an immune response in a subject.

In further embodiments, the adjuvant can be, but is not limited to, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include without limitation MF 59, LT-K63, LT-R72 (Pal et al. Vaccine 24 (6): 766-75 (2005)), QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153, or a less reactogenic composition where the QS21 is quenched with cholesterol as disclosed in PCT publication number WO 96/33739. A particularly potent adjuvant formulation involving QS21 3D-MPL & tocopherol in an oil in water emulsion is described in PCT publication number WO 95/17210. In addition, the nucleic acid compositions of the invention can include an adjuvant by comprising a nucleotide sequence encoding the antigen and a nucleotide sequence that provides an adjuvant function, such as CpG sequences. Such CpG sequences, or motifs, are well known in the art.

Adjuvants can be combined, either with the compositions of this invention or with other vaccine compositions that can be used in combination with the compositions of this invention.

The synthetic tetraspanin protein, nucleic acid molecule, vector, cell, and/or composition of the present invention is intended for use as therapeutic agents and immunological reagents, for example, as antigens, immunogens, prophylactics, therapeutics, vaccines, and/or delivery vehicles. Accordingly, the present invention can be practiced for prophylactic, therapeutic and/or diagnostic purposes. The compositions described herein can be formulated for use as reagents and/or for administration in a pharmaceutical carrier in accordance with known techniques. See, e.g., Remington, The Science and Practice of Pharmacy (latest edition).

Accordingly, another aspect of the present invention provides a method of producing an immune response to an infection in a subject, comprising administering to the subject an effective amount of a synthetic tetraspanin protein, nucleic acid molecule, vector (e.g., extracellular vesicle), population, and/or composition of the present invention.

Another aspect of the present invention provides a method of preventing a disorder associated with an infection in a subject, comprising administering to the subject an effective amount of a synthetic tetraspanin protein, nucleic acid molecule, vector (e.g., extracellular vesicle), population, and/or composition of the present invention.

Another aspect of the present invention provides a method of protecting a subject from the effects of an infection, comprising administering to the subject an effective amount of a synthetic tetraspanin protein, nucleic acid molecule, vector (e.g., extracellular vesicle), population, and/or composition of the present invention.

A “subject” of the invention includes any animal susceptible to a disorder expressing and/or associated with an antigen to which a synthetic tetraspanin protein of the present invention binds (e.g., a cancer antigen, a viral antigen, a bacterial antigen, or any combination thereof). Such a subject is generally a mammalian subject (e.g., a laboratory animal such as a rat, mouse, guinea pig, rabbit, primates, etc.), a farm or commercial animal (e.g., a cow, horse, goat, donkey, sheep, etc.), or a domestic animal (e.g., cat, dog, ferret, etc.). In particular embodiments, the subject is a primate subject, a non-human primate subject (e.g., a chimpanzee, baboon, monkey, gorilla, etc.) or a human. In some embodiments, a laboratory animal may include but is not limited to any standard laboratory mouse strain.

A “subject in need” of the methods of the invention can be a subject known to be, or suspected of being, infected with, or at risk of being infected with, a disorder and/or infection comprising an antigen targeted, expressed, and/or mimicked by a synthetic tetraspanin protein of the present invention (e.g., wherein the synthetic tetraspanin protein of the present invention comprises a heterologous protein or fragment thereof inserted within the second extracellular loop of the tetraspanin backbone wherein the heterologous protein or fragment thereof targets, comprises, and/or mimics the antigen expressed and/or associated with the disorder).

In some embodiments, the subject may be at risk for or suspected to have or develop the infection. In some embodiments, the subject may be at risk for or suspected to have or develop a herpesvirus infection. In some embodiments, the subject may be at risk for or suspected to have or develop a poxvirus infection. In some embodiments, the subject may be at risk for or suspected to have or develop an HIV infection. In some embodiments, the subject may be at risk for or suspected to have or develop a coronavirus infection.

In some embodiments, administering the effective amount of the synthetic tetraspanin protein, nucleic acid molecule, vector, population and/or composition to the subject may comprise administering the effective amount of the synthetic tetraspanin protein, nucleic acid molecule, vector, population and/or composition to the subject prior to the subject developing symptoms of the infection (e.g., comprising administering prophylactically, e.g., as a prophylactic vaccine).

Another aspect of the present invention provides a method of treating to an infection in a subject, comprising administering to the subject (e.g., the subject having or suspected of having or developing the infection) an effective amount of a synthetic tetraspanin protein, nucleic acid molecule, vector (e.g., extracellular vesicle), population, and/or composition of the present invention.

In some embodiments, the infection may be a viral infection. Non-limiting examples of viral infections include infection by a poxvirus, a herpesvirus, human immunodeficiency virus (HIV), and/or a coronavirus (e.g., a common cold, e.g., a SARS-COV-2 infection, e.g., COVID-19 and/or a variant thereof).

Accordingly, another aspect of the present invention provides a method of producing an immune response to a coronavirus infection (e.g., a common cold, e.g., a SARS-COV-2 infection, e.g., COVID-19 and/or a variant thereof) in a subject, comprising administering to the subject an effective amount of a synthetic tetraspanin protein, nucleic acid molecule, vector (e.g., extracellular vesicle), population, and/or composition of the present invention.

Another aspect of the present invention provides a method of preventing a disorder associated with a coronavirus infection (e.g., a common cold, e.g., a SARS-COV-2 infection, e.g., COVID-19 and/or a variant thereof) in a subject, comprising administering to the subject an effective amount of a synthetic tetraspanin protein, nucleic acid molecule, vector (e.g., extracellular vesicle), population, and/or composition of the present invention.

Another aspect of the present invention provides a method of protecting a subject from the effects of a coronavirus infection (e.g., a common cold, e.g., a SARS-COV-2 infection, e.g., COVID-19 and/or a variant thereof), comprising administering to the subject an effective amount of a synthetic tetraspanin protein, nucleic acid molecule, vector (e.g., extracellular vesicle), population, and/or composition of the present invention.

Another aspect of the present invention provides a method of treating to a coronavirus infection (e.g., a SARS-COV-2 infection, e.g., COVID-19 and/or a variant thereof) in a subject, comprising administering to the subject (e.g., the subject having or suspected of having or developing a coronavirus infection) an effective amount of a synthetic tetraspanin protein, nucleic acid molecule, vector (e.g., extracellular vesicle), population, and/or composition of the present invention.

In some embodiments of the methods of the present invention, the synthetic tetraspanin protein (e.g., wherein the heterologous protein or fragment thereof inserted in the synthetic tetraspanin protein) stimulates neutralizing antibodies.

In some embodiments, the synthetic tetraspanin protein (e.g., wherein the heterologous protein or fragment thereof inserted in the synthetic tetraspanin protein) stimulates neutralizing antibodies in an amount equal to or greater than a current commercial vaccine composition comprising the heterologous protein or fragment thereof and/or variant thereof and optionally comprising an adjuvant (e.g., in an amount equal to or greater than a current commercial vaccine composition comprising the heterologous protein or fragment thereof and/or variant thereof and an adjuvant, e.g., alum).

In some embodiments, the methods of the present invention may further comprise co-administering an adjuvant. In some embodiments, co-administering the adjuvant may comprise administering the adjuvant prior to, concurrently with, and/or after administering the synthetic tetraspanin protein, nucleic acid molecule, vector, population and/or composition. In some embodiments, administering the adjuvant concurrently with the synthetic tetraspanin protein, nucleic acid molecule, vector, population and/or composition may comprise administering the synthetic tetraspanin protein, nucleic acid molecule, vector, population and/or composition and the adjuvant as a single administration (e.g., in a single composition). In some embodiments, administering the adjuvant concurrently with the synthetic tetraspanin protein, nucleic acid molecule, vector, population and/or composition may comprise administering the synthetic tetraspanin protein, nucleic acid molecule, vector, population and/or composition and the adjuvant at about the same time, as two separate administrations. As used herein, the term at “about the same time” may comprise any time frame including but not limited to within about 1 minute to about 24 hours of each other, e.g., 1, 2, 3, 4, 5, 6, 7, 8, 9, 10, 11,1 2, 13, 14, 15, 16, 17, 18, 19, 20, 21, 22, 23, 24, 25, 26, 26, 27, 28, 29, 30, 45, 60, minutes, 1, 2, 3, 4, 5, 6, 7, 8, 9, 10, 11, 12, 13, 14, 15, 16, 17, 18, 19, 20, 21, 22, 23, or 24 hours or any value or range therein.

In some embodiments, administering to the subject an effective amount of the synthetic tetraspanin protein, nucleic acid molecule, vector, population and/or the composition may comprise administering a first dosage of the synthetic tetraspanin protein, nucleic acid molecule, vector, population and/or the composition and administering one or more (e.g., two or more, three or more, four or more, etc.) additional dosages of the synthetic tetraspanin protein, nucleic acid molecule, vector, population and/or the composition at least one, two, three, four, five, six, seven, eight, nine, ten, eleven, or twelve or more weeks after administering the first dosage. For example, in some embodiments, the method may comprise administering a first dosage of the synthetic tetraspanin protein, nucleic acid molecule, vector, population and/or the composition and administering a second additional dosage (e.g., a two-dose primary series) of the synthetic tetraspanin protein, nucleic acid molecule, vector, population and/or the composition at least about three weeks after administering the first dosage. In some embodiments, the method may comprise administering a first dosage of the synthetic tetraspanin protein, nucleic acid molecule, vector, population and/or the composition and administering a second additional dosage (e.g., a two-dose primary series) of the synthetic tetraspanin protein, nucleic acid molecule, vector, population and/or the composition at least about four weeks after administering the first dosage. In some embodiments, the method may comprise administering a first dosage of the synthetic tetraspanin protein, nucleic acid molecule, vector, population and/or the composition and administering a second additional dosage (e.g., a two-dose primary series) of the synthetic tetraspanin protein, nucleic acid molecule, vector, population and/or the composition at least about six weeks after administering the first dosage. In some embodiments, the method may comprise administering a first dosage of the synthetic tetraspanin protein, nucleic acid molecule, vector, population and/or the composition and administering a second additional dosage (e.g., a two-dose primary series) of the synthetic tetraspanin protein, nucleic acid molecule, vector, population and/or the composition at least about eight weeks after administering the first dosage. In some embodiments, the method may comprise administering a first dosage of the synthetic tetraspanin protein, nucleic acid molecule, vector, population and/or the composition and administering a second and third additional dosage (e.g., a two-dose primary series and a booster) of the synthetic tetraspanin protein, nucleic acid molecule, vector, population and/or the composition, wherein the second dose is administered at least about two to eight weeks after administering the first dosage, and wherein the third dose is administered at least about one, two, three, four, five, six, seven, eight, nine, ten, 11, or 12 or more weeks after administering the first or second dose, e.g., after administering the second dose (e.g., a two-dose primary series and a booster).

Another aspect of the present invention provides a method of producing an extracellular vesicle comprising a synthetic tetraspanin: delivering a synthetic tetraspanin protein of the present invention and/or nucleic acid molecule and/or vector encoding and/or comprising the same, to a cell capable of producing extracellular vesicles (e.g., contacting the cell, e.g., transfecting the cell), thereby producing a cell producing one or more extracellular vesicles comprising the synthetic tetraspanin; and isolating the one or more extracellular vesicles comprising the synthetic tetraspanin of the present invention.

In some embodiments, the synthetic tetraspanin, nucleic acid molecule, and/or vector may be delivered with a transfection agent (e.g., lipofectamine and the like).

In some embodiments, the method may further comprise immortalizing the cell producing one or more extracellular vesicles comprising the synthetic tetraspanin (e.g., establishing a stable cell line from the cell producing one or more extracellular vesicles comprising the synthetic tetraspanin). In some embodiments, the cell (e.g., the immortalized cell, e.g., the ex vivo cell) may produce extracellular vesicles comprising the synthetic tetraspanin of the present invention, in an amount of at least about 10⁵, 10⁶, 10⁷, 10⁸, 10⁹, 10¹⁰, 10¹¹, 10¹², 10¹³, 10¹⁴, 10¹⁵ or more EVs/ml, or any value or range therein. For example, in some embodiments, the cell may produce extracellular vesicles comprising the synthetic tetraspanin of the present invention in an amount of about 10⁵ to about 10¹⁵ EVs/ml, about 10¹⁰ to about 10¹² EVs/ml, or about 10¹⁰ to about 10²⁰ EVs/ml. In some embodiments, the cell may produce extracellular vesicles comprising the synthetic tetraspanin of the present invention in an amount of at least about 10⁵ EVs/ml, at least about 10¹⁰ EVs/ml, at least about 10¹² EVs/ml, at least about 10¹⁰ EVs/ml, at least about 10¹¹ EVs/ml, or at least about 10²⁰ EVs/ml.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1

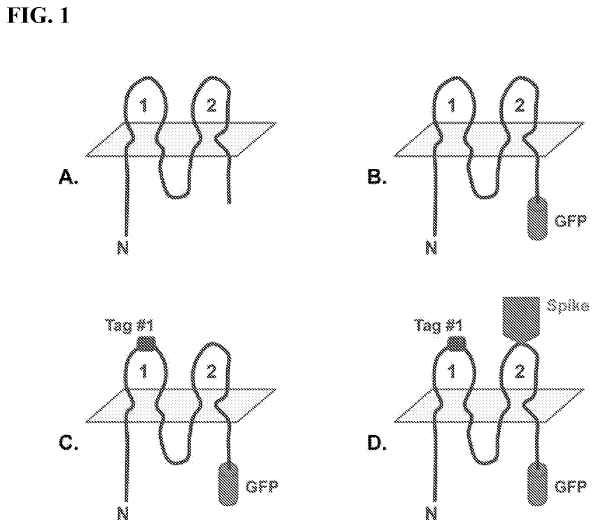

FIG. 1 provides a generic stepwise schematic of the design of the synthetic tetraspanin of the invention. FIG. 1 panel A shows a native molecule with two extracellular loops (1 and 2). FIG. 1 panel B shows an example of the structure modified with a cell-internal, C-terminal tag (e.g., GFP). Such a tag would not be visible to an antibody response. FIG. 1 panel C shows an example of the structure modified with a protein cargo inserted into loop 1, such as for example a short peptide tag of about 5 to about 50 amino acids in length. In some embodiments, a tag may be used for purification of a component (e.g., a vector, a cell, an extracellular vesicle, etc.) comprising the synthetic tetraspanin protein. FIG. 1 panel D shows an example of the structure modified with the insert in loop 1 and/or a second protein inserted into loop 2. In some embodiments, a protein of fragment thereof of about 1200 amino acid sin length, such as but not limited to a SARS-COV-2 Spike protein, may be inserted into loop 2 without affecting expression, folding, and/or trafficking of the synthetic tetraspanin protein and the inserted cargo (e.g., the inserts of loop 1 and/or loop 2).

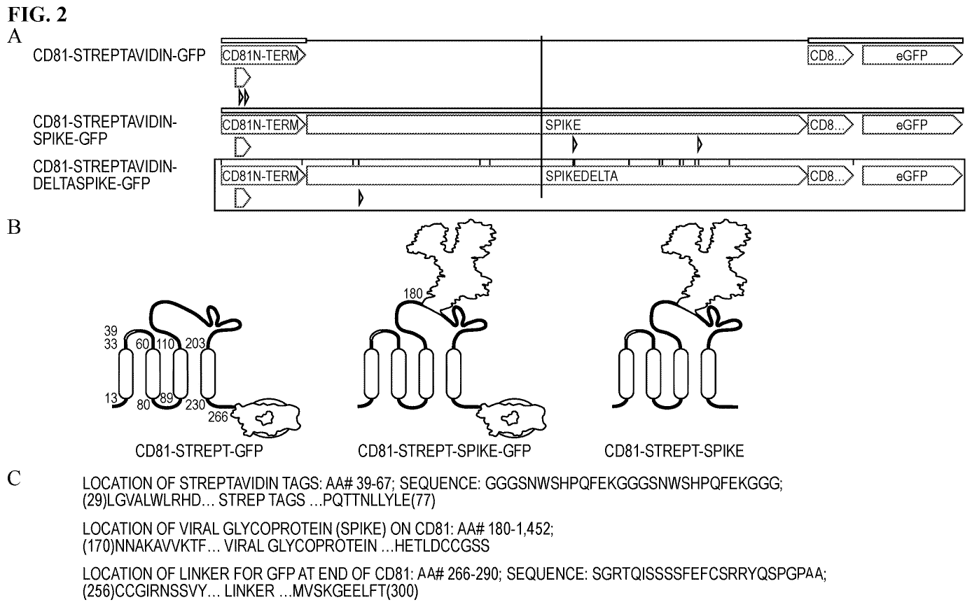

FIG. 2 illustrates an example construction of a delivery expression vector. While not wishing to be bound to theory, successful insertion of a protein sizing about 1200 amino acids has not previously been reported in a backbone such as a tetraspanin (236 amino acids), as most insertions disrupt protein folding and/or trafficking of either the backbone recipient (e.g., the tetraspanin) or the cargo (e.g., the Spike insert, as exemplified in FIG. 2). FIG. 2 panel B provides a schematic of the position of the four transmembrane domains, and shows where an example cargo such as a SARS-COV-2 Spike domain is inserted. FIG. 2 panel C shows the particular positions of the insertions in the tetraspanin backbone as shown in FIG. 2 panel B.

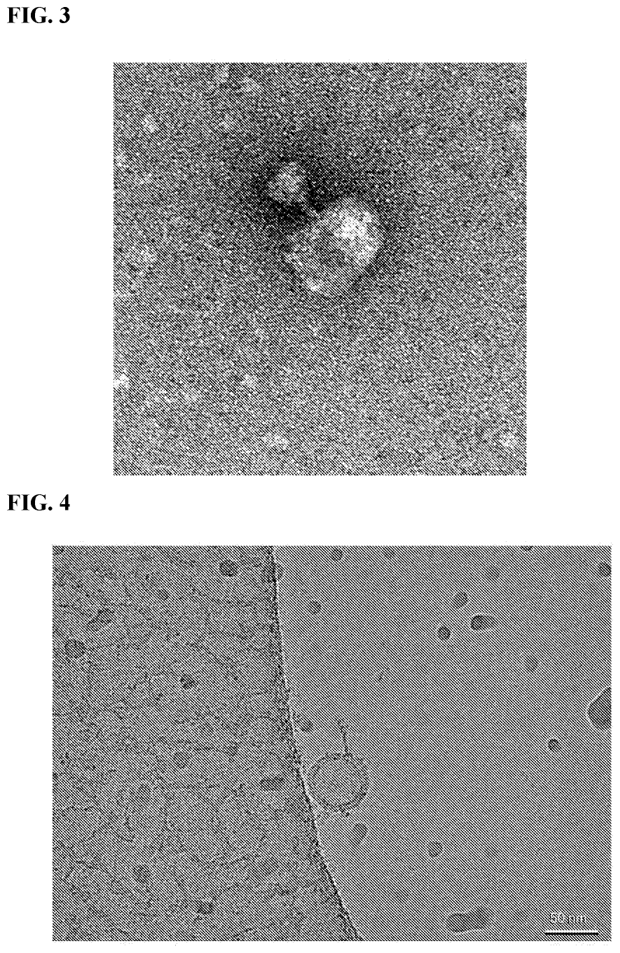

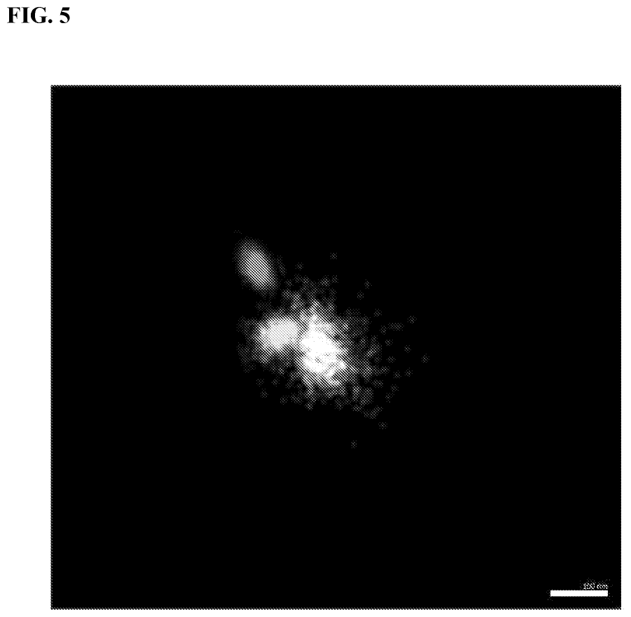

Studies were performed to identify whether the inserted cargo (e.g., a SARS-COV-2 Spike protein) is properly expressed and folded when incorporated into an exosome using the synthetic tetraspanin of the present invention. Data shown in FIGS. 3 and 4 demonstrate expression, localization into EVs and folding by electron microscopy and cryo-election microscopy. FIG. 3 shows an electron micrograph depicting a purified exosome showing the structure for an exosome, which is round, and SARS-COV-2 Spike, which is folded as a trimer. Both the exosome and the Spike protein are of expected size. FIG. 4 shows the Spike protein recognizable by its elongated shape and fork-like folding, emerging perpendicular to the membrane of the vesicle. While not wishing to be bound to theory, this extended and exposed localization is understood to be important for inducing a proper antibody response. The length of the Spike stalk was determined to be 25 nm, which is consistent with the image and approximately equal to the radius of the EV.

FIG. 5 shows an immunofluorescence micrograph of a single EV (top left) and probed with an antibody to anti-SARS-COV-2 Spike (bottom right) and anti-CD81 antibody (bottom left). 90% of CD81-positive EVs had Spike on them. The EVs without Spike typically did not have CD81. The three molecules clearly colocalize within 100 nm (see scale bar). This data demonstrates proper trafficking of the tetraspanin construct into EVs, stability throughout purification, and proper folding under physiological conditions, evidenced by the antibody epitopes remaining preserved.

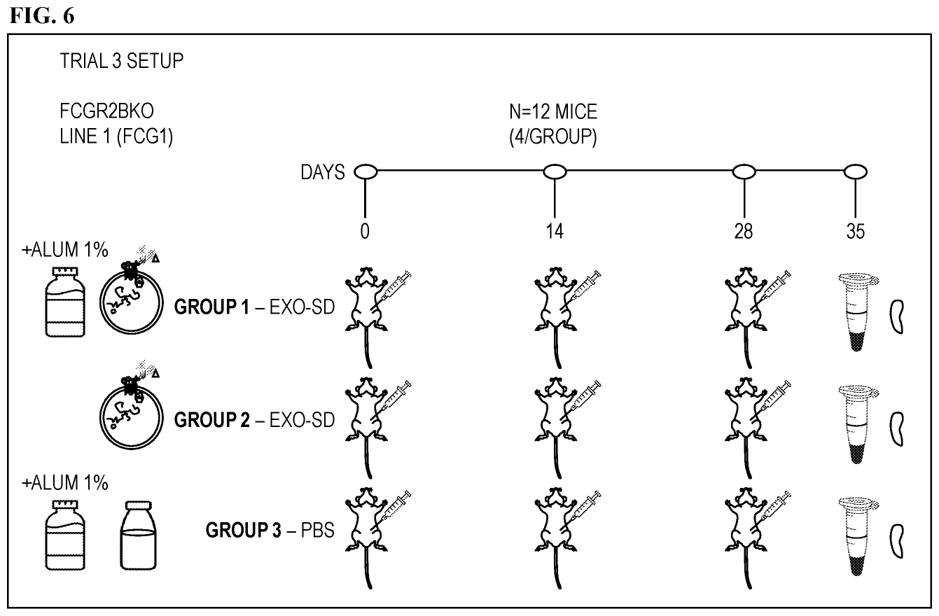

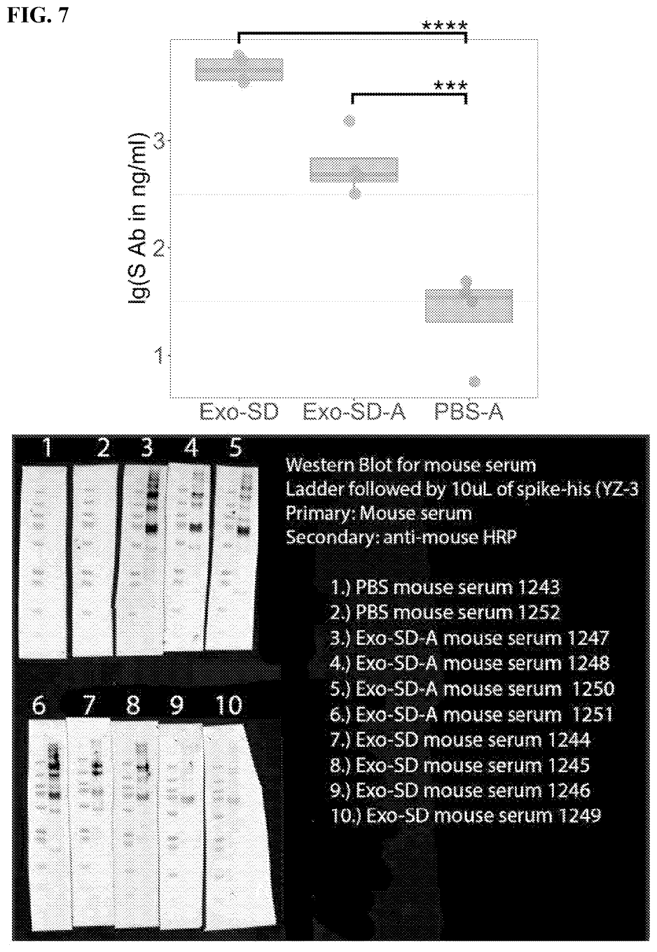

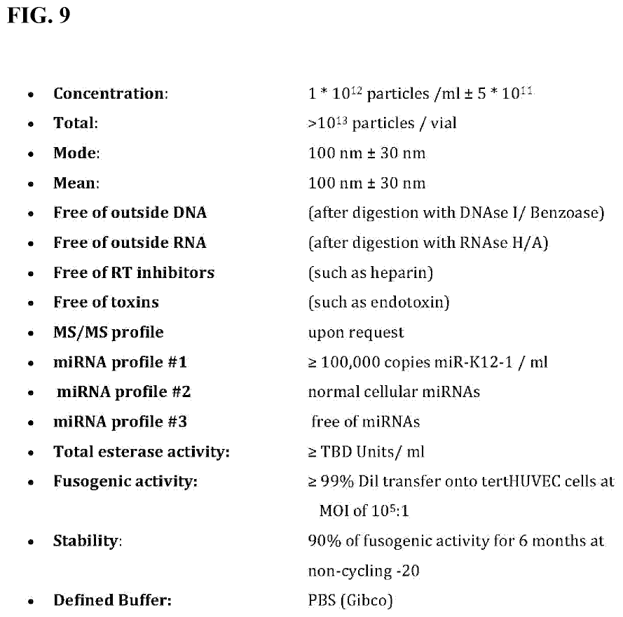

Experiments were performed to analyze the immune reactivity upon immunization using the synthetic tetraspanin of the present invention, with or without adjuvant (FIG. 6). The experimental design included three groups of n=4 each. The three groups were Exo-SD plus 1% alum as an adjuvant, Exo-SD without adjuvant, and PBS plus 1% alum. Mice were immunized three times, replicating the CDC-recommended vaccination schedule for subunit vaccines in persons at high risk of COVID disease. One week after the last boos, serum and spleen samples were harvested and analyzed. Results are shown in FIGS. 7 and 8. As shown in FIG. 7, SARS-COV-2 Spike/CD81 constructs delivered by EVs without adjuvant produced a superior immune response as compared to alum-adjuvanted EVs. This response was specific for the trimeric, correctly folded form of SARS-COV-2 Spike as determined by ELISA. FIG. 8 shows the results of western blot assays using mouse antisera to recognize bacterially expressed SARS-COV-2 Spike. Spike exists in monomeric and trimeric forms, and perhaps lower molecular weight breakdown products as well. The sera obtained from mice immunized with PBS alone did not recognize any bands (e.g., bands labeled #1 and #2). FIG. 9 lists particulars of example isolated EVs comprising a synthetic tetraspanin of the present invention.

### Example 2: Extracellular Vesicles Engineered into Antigen Presentation Platforms

Creation of Constructs: The CD81-Spike recombinant plasmids were designed by extracting sequences from the indicated database and built into the pcDNA3.1 (+) plasmid: CD81 (NCBI, CCDS, 7734.1), GFP (Addgene Plasmid 62964), Spike (GenBank, MT565498.1), Spike A variant (GISAID, Accession ID EPI_ISL_2710011, add G142D, R158G and D950N). Stabilized spike A variant was built by making the proline stabilizing mutations (K986P, V987P, F817P, A892P, A899P, A942P) (33) into the Spike A variant sequence. Plasmids were synthesized by Genscript and confirmed by digestion and sanger sequencing. The constructs used in this study are Spike-GFP (pDD3511), CD81-Strep-GFP (pDD3513), CD81-Strep-Spike-GFP (pDD3515), CD81-Strep-A Spike-GFP (pDD3519), CD81-Strep-Stabilized A Spike-GFP (pDD3521). A secreted form of stabilized Spike protein expression plasmid (pDD3503) also used (Stadbaeur et al. 2020 Curr Protoc Microbiol. 57 (1): e100).

Cell Culture: Human osteosarcoma cells (U2OS) were obtained directly from the ATCC (HTB-96) and certified as mycoplasma free. Cells were grown in Dulbecco's Modified Eagle Medium (DMEM) (Thermo Fisher 21013024) supplemented with 10% exosome-free Fetal Bovine Serum (FBS) (VWR 97068-085) and 100 units/mL of Penicillin, 100 μg/mL of Streptomycin solution (Gibco 15140-122). Cells were maintained at 37° C. and 5% CO₂ in the HERAcell 150i incubator (Thermo Fisher 50116050).

After the creation and validation of the constructs, U2OS cells were transfected with the construct of choice using Lipofectamine-2000 (Thermo Fisher 11668019) diluted in OptiMEM. After 24 hours, the cells were placed in mammalian selection (G418 at 250 μg/mL). After selection, single cell sorting using a FACS Aria II BSL2. These clones were grown to create a homogeneous population to decrease heterogeneity in the EVs produced.

Purification of EVs: After sorting, cells were seeded and grown to 80% confluency, then conditioned with complete media. After 72 hours, conditioned media was collected and centrifuged at 1000×g for 5 minutes. Next, media was filtered through a 0.45 μm (Genesee, Cat. #25-230) and a 0.22 μm filter (Genesee, Cat. #25-229). The media was then concentrated by tangential flow filtration (TFF). An ÄKTA Flux Tangential Flow Filtration System with a MidGee Hoop hollow fiber (Cytiva, 750 kDa MWCO, UFP-750-C—H24LA) was equilibrated in sterile 1× phosphate buffered saline (PBS, Gibco, Cat: 14190144). Ultrafiltration to a final volume of 45 mL was completed using a feed flow of 34 mL/min at a TMP of 27 psi. Concentrated media was then incubated with a 4% final concentration of polyethylene glycol (PEG, Fisher BioReagents, Cat. #BP233-1) overnight at 4 °C. The next day, the media was centrifuged at 1000×g for 1 hour at 4 °C. The supernatant was removed, and the crude PEG pellet was resuspended in cold PBS with RNase (ThermoFisher, Cat #EN0531) and DNAse (Promega, Cat #: M6101) and incubated overnight at 4 °C. Finally, the resuspension was run through a flowthrough method using a HiTrap™ Capto Core 700 column (Cytiva, cat: 17548151). The EVs were kept at −80° C. until use.

Western Blot Analysis: Cell lysates were lysed in RIPA buffer and EVs were lysed in a strong lysis buffer. Protein levels were determined using a BCA kit. Lysates were run on a precast SDS gel. After transfer, the membrane was stained with Ponceau to check for total protein. The membrane was then blocked in 5% milk in TBST for 1 hour and put in a primary antibody overnight in 5% BSA in TBST. The next day, the secondary antibody was diluted in 5% milk in TBST and imaged with an Odyssey fluorescence or an iBright for chemiluminescence. The following antibodies were used: GFP, CD81, CD63, Actin, Spike, Flotillin-2, Syntenin-1, anti-mouse Licor, anti-rabbit Licor, anti-rabbit HRP, and anti-mouse HRP.

Electron Microscopy: For transmission electron microscopy (TEM), total and affinity-purified EVs were adsorbed on a glow-charged carbon coated 400-mesh copper grids for 2 minutes and then stained with 2% (weight/volume) uranyl acetate in water. TEM images were taken using a Philips CM12 electron microscope at 80 kilovolts. Images were captured on a Gatan Orius camera (2000×2000 pixels) using the Digital Micrograph software (Gatan, Pleasanton, CA). Zoomed-in images were created and adjusted in Adobe Photoshop. For cryo-EM, samples were absorbed on a glow-charged grids (Quantifoil R 1.2/1.3, 400 Mesh, Copper, Cat. Q425CR1.3, EMS) for 30 seconds and blotted for 2 to 4 seconds to remove extra samples. Then the grids were snap froze in ethane/propane pre-chilled to −165° C. and imaged using Thermo Fisher Scientific Talos Arctica G3.

Fluorescence Microscopy: Cells were seeded onto glass coverslips inside a six-well plate (Fisher 07-200-83) and allowed to grow for 24 hours. Plasmids were then introduced into the cell via transient transfection with Lipofectamine-2000 (ThermoFisher 11668019) at a 1:2 ratio of plasmid to Lipofectamine. The cells were allowed to grow for another 24 hours, fixed with 4% paraformaldehyde in PBS for 15 minutes at room temperature, and washed with 0.1% TBST. Cells were then blocked with 5% BSA in 0.1% TBST for 30 minutes at room temperature.

Primary antibodies were diluted in 5% BSA in TBST and allowed to incubate with coverslips for 3 hours at room temperature or 4° C. overnight. Coverslips were then washed with 0.1% TBST. Secondary antibodies were diluted in 5% BSA in TBST and allowed to incubate with coverslips for 1 hour at room temperature. Cells were washed and 4′,6-diamidino-2-phenylindole (DAPI) was diluted to a concentration of 0.01% in water and added to the wells for 5 minutes. Coverslips were then washed with water and mounted onto Frosted Micro Slides (Corning 2948-75×25) using 50 μL of ProLong Gold Antifade Reagent (Cell Signaling 9071S).

The slides were imaged using the DM5500 microscope (Leica) using the Leica HC PL Apo 100× Oil Objective. 2D-deconvolution was then performed on the Z-stacks using MetaMorph V 7.8.12.0. Images were visualized and edited in Image J 1.8.0_172.

Super Resolution Microscopy: Glass-bottom 15 u-slide 8 well plates (Ibidi 80827) were prepared by adding 0.01% Poly-L-Lysine to each well overnight at 4° C. The affinity-purified and Cell Mask Red (Thermo Fisher C10046) stained EVs were placed into the Poly-L-Lysine coated wells in a total volume of 1E10 EVs in 200 μL PBS per well and allowed to adhere to the surface overnight at 4 °C. 0.05% paraformaldehyde in PBS was added to each well and allowed to incubate for 30 minutes at room temperature. The solution was then carefully removed with a pipette to not disturb the EVs, and EVs were washed with PBS. EVs were then blocked with 5% BSA in PBS for 30 minutes at room temperature before antibody labeling.

Antibodies were then conjugated to a photoswitchable fluorophore using an Alexa Fluor antibody labeling kit, according to the manufacturer's protocol. The CD81 antibody (Invitrogen #MA5-13548) and S1 spike antibody (Invitrogen #PA5-114446) were labeled with the Alexa Fluor 488 (Thermo Fisher A20181) and Alexa Fluor 568 Labeling Kits (Thermo Fisher A20184), respectively. Additionally, purified ACE2-His protein (Sino Biological 10108-H08H-B) was conjugated using the Alexa Fluor 568 conjugation kit. The first antibody was then diluted in 5% BSA in PBS, and 150 μL were added to each well for 2 hours at room temperature. The antibody solution was removed, and wells were washed with PBS. The blocking and antibody labeling steps were then repeated with the second antibody. B-cubed buffer was then prepared to a 0.05% concentration of enzyme protocatechuate dioxygenase in imaging buffer (ONI, BCA0017) and added to each well 30 minutes before imaging to scavenge oxidizing molecules.

The Nanoimager (Oxford Nanoimaging) was calibrated for dSTORM using 100 nm Tetraspek microspheres (Invitrogen T7279) diluted to 1% in water and placed into Glass Bottom 15 u-Slide 8 well plates. 3-D mapping calibration and channel mapping calibration were completed to obtain the X, Y, and Z-axis errors.

The EVs were then viewed using a custom 405/473/561/640 nm excitation laser configuration (ONI Nanoimaging). During image acquisition, the laser power was raised by 3 increments of 10 every 1000 frames or raised enough to maintain a high signal-to-noise ratio while preventing photobleaching of the fluorescent markers.

During image analysis, post-acquisition correction was performed on the unfiltered image. Photon count, localization precision, sigma, frame index were adjusted as described in (35). Data was then analyzed using the CODI program (ONI Nanoimaging). Colocalization data was exported to excel, and pie charts were made in R 4.2.1 using the ggplot2 package.

Mice: FCGR2bKO Line 1 mice (FCG1) were generated by the UNC Animal Models Core facility (AMC) using BALB/cJ zygotes to establish founders with a 92-bp deletion in the FCGR2b allele. These were mated to wild-type BALB/cJ females to establish a colony. The mice were subsequently bred to homozygosity for the nonfunctional allele, which was detected by PCR with primers Fcgr2b-3ScF1 (5′-CCTGCTGAGGATCAATTACACTC-3′; SEQ ID NO: 9) and Fcgr2b-3ScR1 (5′-GGATGCTTCCCAGAAACCA-3′; SEQ ID NO:10). A 496 bp band for the Fcgr2b deletion allele and a 588 bp band for the wild-type allele was detectable.

Mice were maintained and bred in a pathogen-free Animal Biosafety Level 1 (ABSL1) facility. Experimental manipulations took place in an ABSL2 facility, where mice were housed under aseptic conditions.

Administration of EVs in mice: To maintain sterility, EVs were thawed to RT without external heat inside the biosafety cabinet. Injection preparations were made by diluting EVs to le10 particles/mL in sterile PBS or with aluminum hydroxide (alum) gel, also known as Alhydrogel (InvivoGen VAC-ALU-250), to a final concentration of 1% in PBS. Alhydrogel is a well-described FDA-approved vaccine adjuvant. EVs were thoroughly mixed in the biosafety cabinet by pipette. Injections containing alum were mixed for at least 5 minutes to adsorb the antigen, following manufacturer instructions.

The EVs were prepared same-day and given to the ASC for subcutaneous injection at a volume of 100 μL per animal. Mice (n=12) were divided into three treatment groups of 4 mice each: EVs with alum, EVs without alum, and PBS with alum. There were three injections given in total, fourteen days apart. Seven days after the final injection, the mice were terminated for sample collection. Whole blood was collected via cardiac puncture, and spleens were harvested and fixed in 10% formalin for 24 hours at 4 °C.

Serum Collection and Downstream Analysis: The whole blood was collected into untreated sterile microcentrifuge tubes and was allowed to clot upright for 30 minutes at room temperature. Serum was then separated by centrifugation at 4° C. and 7500 × g for 15 minutes. The serum was collected, diluted 1:1 with PBS, and incubated in a 56° C. dry bath for 30 minutes to inactivate complement. Antibody levels were determined using Mouse Anti-SARS-COV-2 Antibody IgG Titer Serologic Assay Kit (Spike trimer) from Acro Biosystems (RAS-T023). The enzyme-linked immunosorbent assay (ELISA) was performed according to manufacturer instructions using a final serum dilution of 1:1000. Positive and negative controls were similarly diluted 1:1000. Mouse anti-SARS-COV-2 IgG standards provided by the manufacturer were serially diluted to create a standard curve for quantification.

Samples for the ELISA were plated in duplicate, and washes were performed using a BioTek ELx405 microplate washer. Absorbance was detected as optical density (OD) using a CLARIOstar Plus microplate reader (BMG Labtech) at 450 nm with 630 nm as the reference wavelength. Titers for serum samples were determined by applying the appropriate reference and blank corrections, then plotting the duplicate average on the standard curve to obtain the calculated sample concentration. Multiplying by the dilution factor yielded the serum antibody concentration in ng/ml.

Purification of Spike-His: The Spike-His construct was transfected into U2OS cells. After 48 hours, the media was harvested by spinning at 1000 × g for 10 minutes, then filtered through a 0.45 μm filter. Protease inhibitor was added to the filtered media to prevent spike degradation, and 20 mL of 20 mM sodium phosphate, 10 mM imidazole, 300 mM sodium chloride, pH 7.4 was mixed into the media. The media was then run through a HisTrap™ HP column (Cytiva, Cat: 29051021) for affinity purification of the histidine-tag of the spike protein. The column was washed with 20 mM sodium phosphate, 25 mM imidazole, 300 mM sodium chloride, pH 7.4. The spike-his construct was then eluted with an elution buffer of 20 mM sodium phosphate, 500 mM imidazole, 300 mM sodium chloride, pH 7.4. The Spike-His was then kept in-80° C. until use.

Flow Cytometry: BD Accuri C6 Plus Flow Cytometer was calibrated with 2 drops of CS&T RUO beads diluted in 500 μL nanoparticle water (BD Biosciences, Cat: 661414). Gatings were created to select singlets and live cells based on FSC-A X FSC-H and FSC-A X SSC-A, respectively. YZ13/15 CD81 gRNA cell lines were reported with CD81 protein fluorescence using an FITC optical filter. U2OS wild type (WT) cells were used as control.

Upon confluency, CD81-GFP, CD81-Spike-GFP, and U2OS WT cells were rinsed with Dulbecco's Phosphate Buffered Saline (Gibco, 14190-144) of equivalent media volume, followed by 0.05% Trypsin-EDTA (Gibco, ThermoFisher, P: 25300-054) through 5 min of incubation at 37 °C. Treated cells were added back to 10 mL of media. A total of 1 mL lifted cells were saved for fluorescence reading using flow cytometer. Clones with a single peak of FITC-H+ count were selected. Each selected colony underwent a total of 3 readouts.

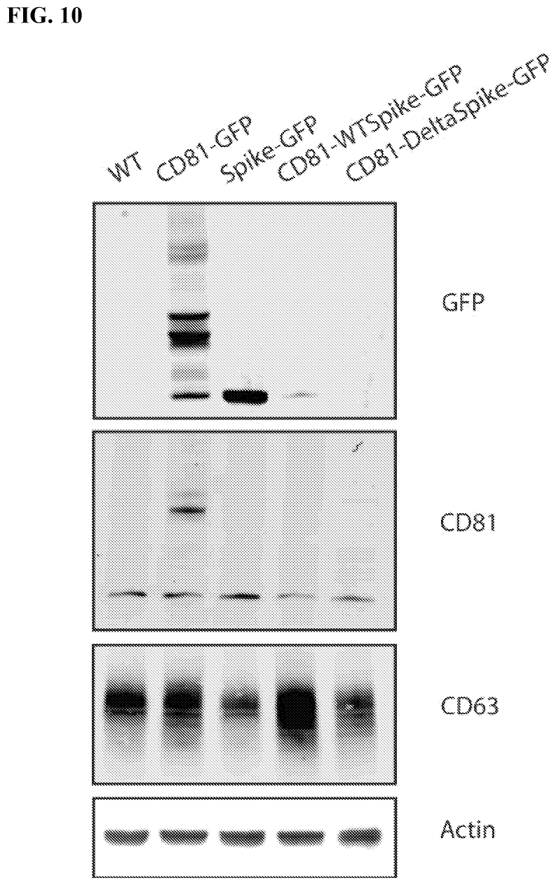

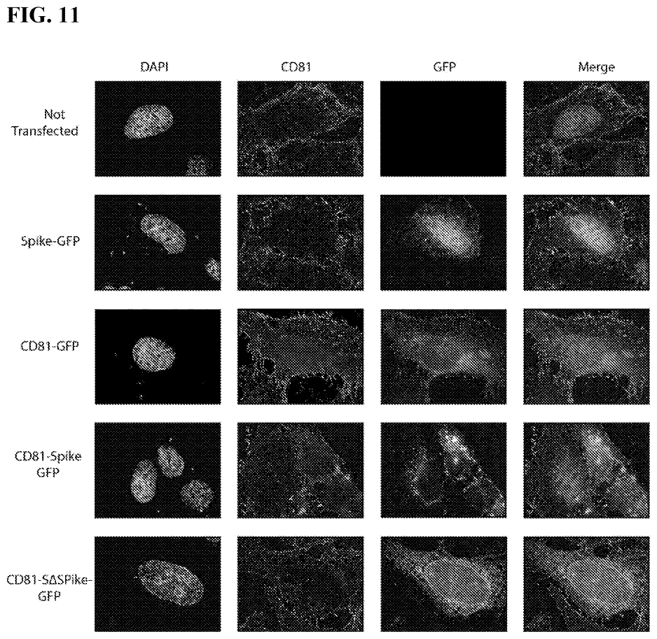

Results Creation of constructs: Tetraspanin protein formation includes two extracellular loops, the small extracellular loop (SEL) and the large extracellular loop (LEL). Since the LEL of CD81 sits on top of the SEL, it was determined that Spike would be the most accessible on the LEL. As seen in FIG. 2, a CD81-GFP construct was generated and a WT Spike or a proline-stabilized A Spike inserted the LEL. Flow cytometry to determine the GFP signal in the cell. A western blot was performed to validate these cell lines, as seen in FIG. 10. The WT cell line was negative for GFP. The CD81-GFP had two CD81 bands, an endogenous and a mutated version, which can also be seen with the GFP antibody. The Spike-GFP cell line also had a thick GFP band around 25 kDa. While not wishing to be bound to theory, it was hypothesized that the GFP is cleaved off Spike since there are no stabilizing substitutions. Cleaved GFP was also seen in the non-stabilized CD81-WT Spike construct. However, the stabilization of A Spike on the CD81 had no visible cleaved GFP. Since Spike was on the CD81 LEL, where the antibody binds, other CD81 bands other than the endogenous expression were not seen.

Spike has an altered localization when added to CD81: The intracellular localization of GFP in these constructs was next determined. A CD81 antibody was used as a membrane marker (Mathieu et al. 2021 Nat Commun 12 (1): 4389) and to see any colocalization (FIG. 11). As seen in the non-transfected cells, there was no GFP signal, and CD81 was enriched on the plasma membrane. In the Spike-GFP construct, a perinuclear GFP signal was seen. Since the GFP seemed to be cleaved off quickly (FIG. 10), the GFP may also be quickly degraded, leading to most of the GFP signal being the newly created GFP. In the CD81-GFP construct, GFP was found throughout the cell and had colocalization with the CD81 antibody. When WT Spike and A Spike were added to CD81, GFP appeared to leave the nucleus, with a signal similar to that of the CD81-GFP cells (FIG. 11).

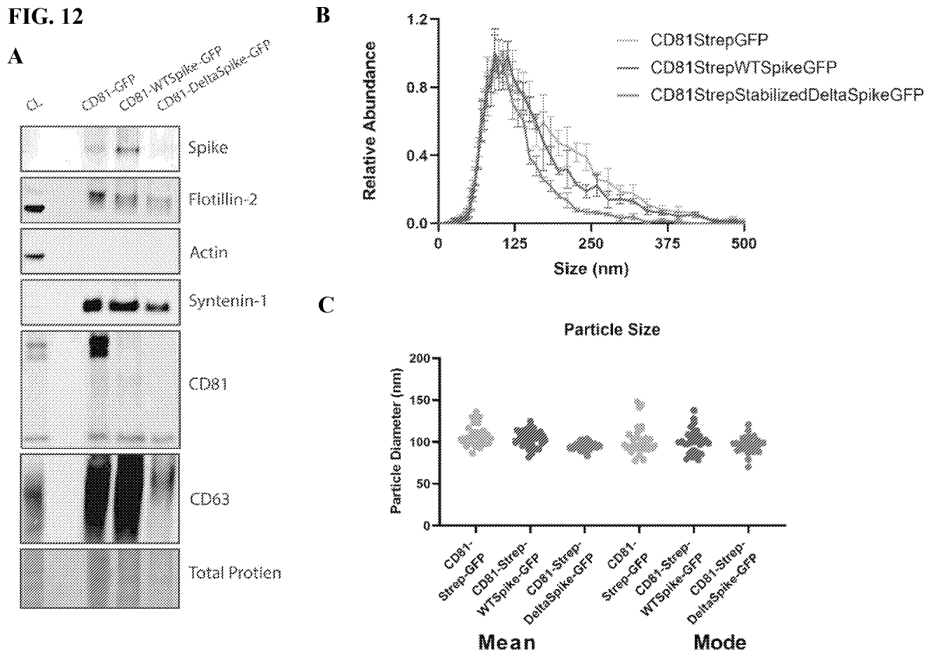

Spike is packaged into EVs and does not change EV size: After determining the constructs altered spike localization, it was next determined if CD81-Spike is packaged into EVs. Therefore, culture media was harvested and EVs were purified the EVs. The EVs were enriched in typical EV biogenesis markers (FIG. 12). Using a WT S1 antibody, a Spike band was found in the CD81-WT Spike-GFP construct. However, this Spike band was not seen in the A construct, which was hypothesized to be because the S1 is specific for the WT Spike and does not bind to the mutated region of the A Spike. Additionally, no significant differences were found in the mean or mode size of the EVs (FIG. 12 panels B and C).

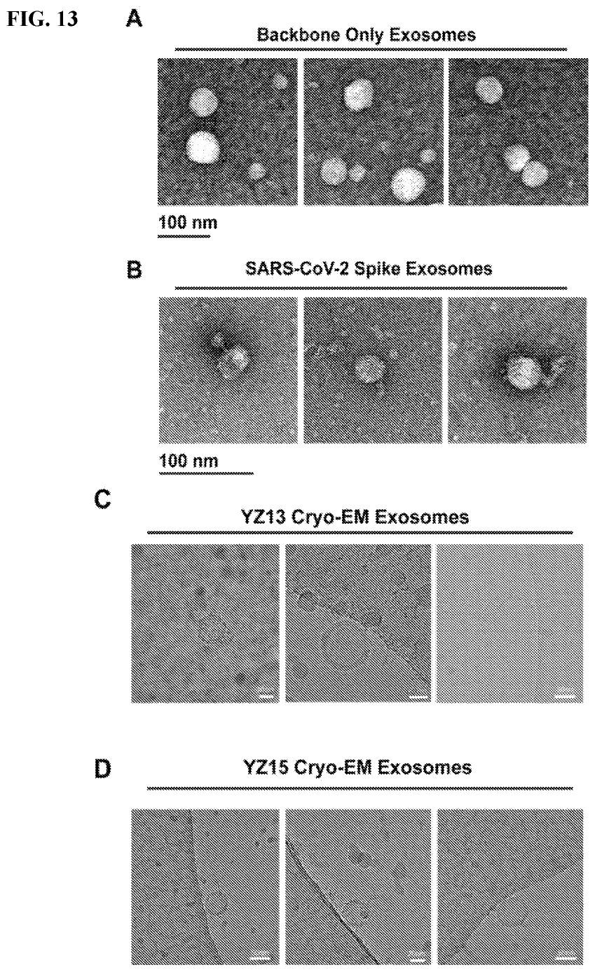

Spike can be visualized on the EVs: Transmission electron microscopy (TEM) and cryo-EM were used to visualize the purified EVs. First, TEM was used to visualize that the CD81-GFP EVs were around 80 nm in size and round (FIG. 13 panel A). However, in the CD81-Spike-EVs, a similar sized but club-shaped protein was seen outside the EV (FIG. 13 panel B). To get a better visualization of these EVs, cryo-EM was used to look at the EVs. Similar to the TEM results, round, small EVs were seen in the CD81-GFP with the double membrane (FIG. 13 panel C). The distinct Spike morphology was also visible on the CD81-Spike-GFP EVs (FIG. 13 panel D).

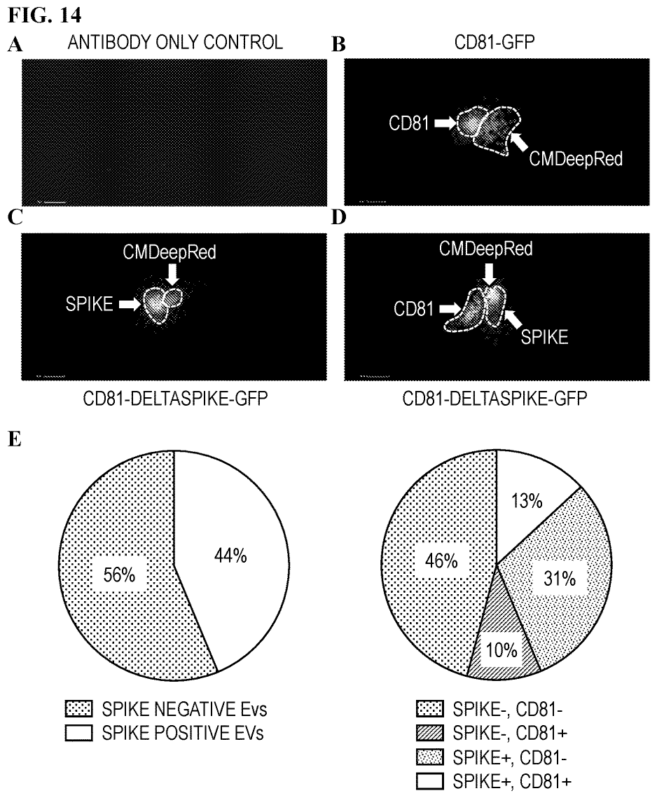

In addition to using EM, direct stochastic optical reconstruction microscopy (dSTORM) was used to image individual EVs. The backbone-only EVs and the A Spike EVs were stained for CD81 attached to an AlexaFluor 488 and Spike S1 attached to an AlexaFluor 564. The EVs were also stained with CellMask deep red, which stains the membrane of the EVs. The EVs were first stained with the CD81-488 for 2 hours, washed, then stained with the Spike-564 for an additional 2 hours. Minimal background staining in the EV negative well was found (FIG. 14 panel A). Additionally, the Spike antibody did not bind to the Spike-negative EVs (FIG. 14 panel B). The Spike antibody bound well to the EVs expressing the stabilized Spike (FIG. 14 panel C). Interestingly, some EVs had both CD81 and Spike staining (FIG. 14 panel D). Since the CD81 antibody cannot bind to the LEL of the CD81-Spike-GFP, the CD81 stained was likely endogenous, and that there can be multiple CD81 molecules per EV. 44% of the EVs expressed Spike. Of the 56% that did not express, only 10% were positive for CD81 only (FIG. 14 panel E). These results suggest that 46% of the EVs may be made through a separate pathway that does not require CD81, such as described in Mathieu et al. To increase the amount of Spike-positive EVs, knocking out other biogenesis proteins, such as CD63, may enhance the production of CD81-positive, and therefore Spike-positive, EVs.

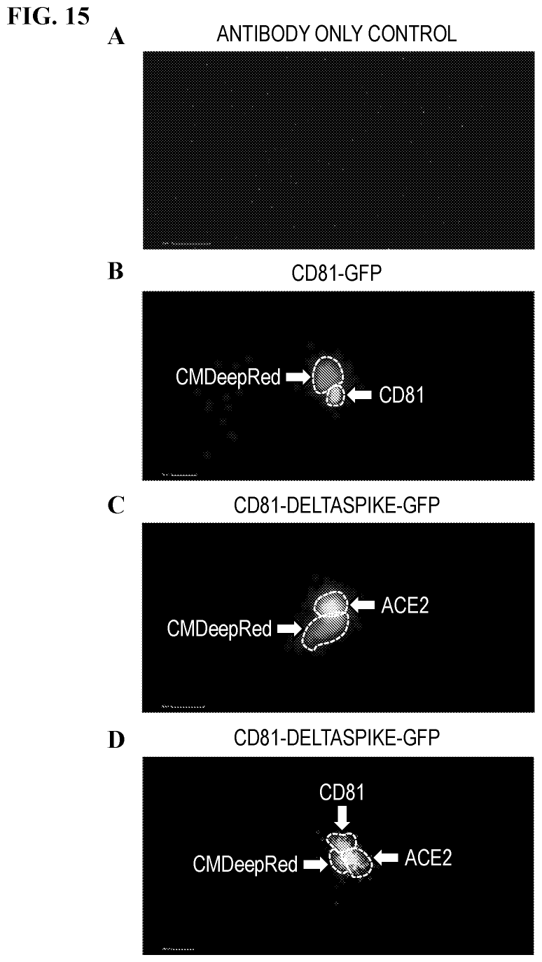

The Exo-Spike construct can bind to ACE2: When the Spike protein trimerizes in the virion, the receptor-binding domain (RBD) gets pushed up to better interact with ACE2. To determine if the Spike on the surface of the EVs could also bind ACE2, purified biotinylated ACE2 was used to attach an AlexaFluor-568. Similar to the experiment in FIG. 14, an EV negative well, the CD81-GFP EVs, and the CD81-Stabilized A Spike-GFP EVs, were stained first with CD81-488 for 2.5 hours, then with ACE2-568 for 2.5 hours. There was some background staining in the antibody only well, but very little colocalized with each other (FIG. 15 panel A). The backbone construct had virtually no ACE2 staining (FIG. 15 panel B). Finally, the Exo-Spike EVs was found to bind ACE2 (FIG. 15 panel C), and there were many triple positive EVs (FIG. 15 panel D).

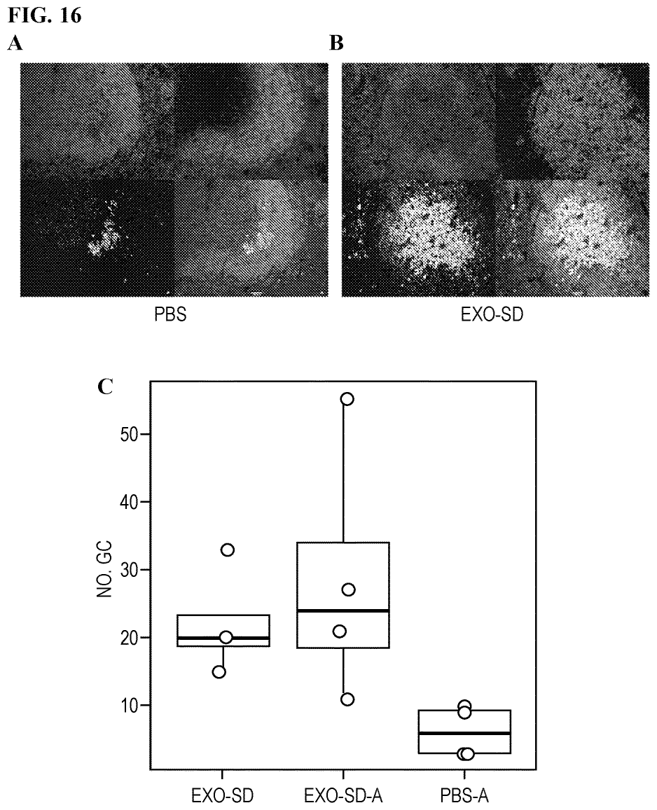

Spike-expressing EVs elicit an immune response in mice: 1E10 EVs or 100 μL of PBS were injected into mice three times, fourteen days apart, as shown in FIG. 6. Alum was used in two groups. WT-Spike construct EVs were tested first, but it was discovered there were not many neutralizing antibodies and no trimer antibodies present. However, using a stabilized A-Spike construct, it was found that treatment with EVs induced many spike antibodies in the mouse serum (FIG. 7). Additionally, purified Spike protein was run on a western blot using the serum from the mice in place of a primary antibody. It was found that the serum from the mice injected with the Exo-Spike and Exo-Spike with Alum could bind to the purified Spike, but the serum from mice treated with PBS could not bind (FIG. 8). The spleens of the treated mice were also stained for germinal centers (GC). Production of antibody-producing B-cells or plasmacytes occurs in the GC and is positive for B-cell marker B220 (magenta) and BCL-6 (yellow), as shown in FIG. 16. The PBS also had GC; however, they seemed smaller on average (FIG. 16 panel A). The GC was more prominent in the Exo-SD treated mice (FIG. 16 panel B). The number of GC and size were quantified, and differences were differences between the PBS and Exo-SD groups, though not significant (FIG. 16 panel C). No adverse side effects of the Exo treatment, such as weight loss, were found.

### Example 3: Exosome-Based SARS-COV-2 Spike Immunization Experiments

The first cluster of SARS-COV-2, leading to the COVID-19 pandemic, was reported in late 2019. The virus is a positive-sense single-stranded RNA virus with an enveloped helical nucleocapsid. The primary target of COVID-19 vaccines is the Spike(S) protein of the virus, especially the receptor-binding domain (RBD). The S-RBD domain interacts with the human angiotensin-converting enzyme (ACE2) in the respiratory tract to facilitate virus entry. There are also ways for SARS-COV-2 to enter cells that are ACE2 negative.

There are four main vaccines based on selected expression of the SARS-COV-2 Spike protein. Two mRNA-based (mRNA-1273 and BNT162b2), one viral vector (Ad26.COV2.S), and one protein-based adjuvant (NVX-CoV2373 in clinical trial). The vaccines targeting the S RBD domain block spike-ACE2 fusion and induce the most effective neutralizing antibodies. They do not prevent viral transmission, but severe disease. Other vaccines use other parts of the virus or even the entire, inactivated virus, such as the VALNEVA vaccine or COVAXIN. It is unknown in what form the Spike protein is present on these vaccines and how many of the particles are effective. Vaccines aimed at other Spike regions, like S1 or S2, might not produce neutralizing antibodies but can still offer protection and enhance cross-protection.

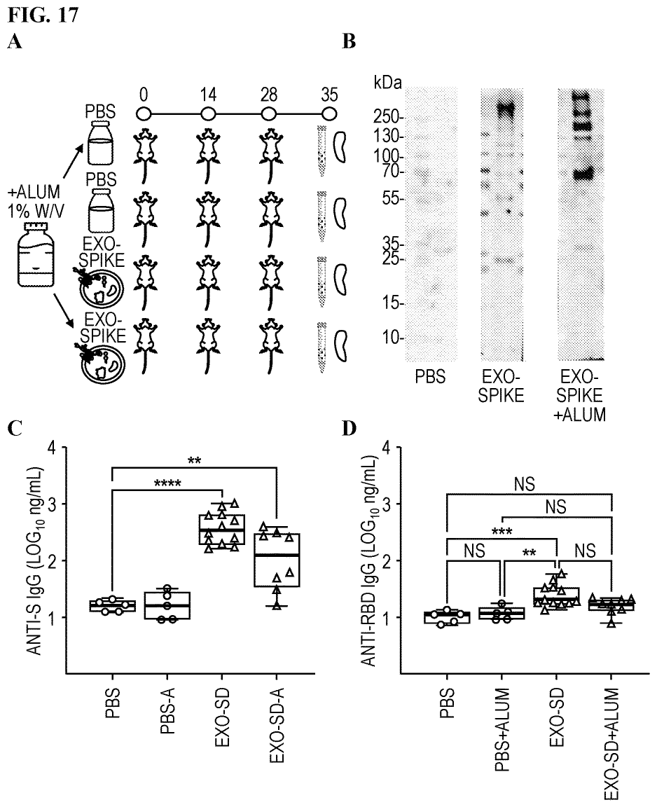

As shown in FIG. 17, experiments were performed to examine a treatment plan and boost regimen. Numbers on top indicate days of injection or (day 35) collection. Alum at 1% w/v was added as a carrier in some groups. The group size was n=5. “Exo-Spike” refers to the invention as described herein. “PBS” refers to negative control or saline (FIG. 17 panel A). Western-Blot assays were performed to test sera for the presence of anti-SARS-COV-2 Spike antibodies (FIG. 17 panel B). The target Spike-His protein (construct pDD3503) was collected from conditioned cell media and purified to near homogeneity using a His column. Protein was run on an SDS-PAGE gel. Serum from mice injected with “PBS”, “Exo-Spike”, or “Exo-Spike” alum was incubated with the membranes at 1:100 dilution and detected with anti-murine total IgG-conjugated to horseradish peroxidase. Dark bands indicate that serum antibodies from the mice recognize the full-length SARS-COV-2 spike protein and its break-down products. Weak bands on the left and numbers indicate molecular weight markers in kDa.

Results of an ELISA assay testing for the presence of SARS-COV-2 trimer-Spike antibodies are shown in FIG. 17 panel C. Trimeric Spike, rather than monomeric Spike is the native species that recognizes the human ACE-2 receptor and mediates virus entry. Hence, this assay is a surrogate for protective immunity. This uses a commercial ELISA (Acro Biosystems RAS-T023). Shown is a box and whisker plot of range, median, 1st and 3rd quartile overlayed with individual data points for either S negative group (circles) or the S positive group (triangles). The amount of anti-S specific IgG is shown on the vertical axis in ng/ml on a log 10 scale. Significance is indicated by the number of starts with *: p<0.05, **: p≤0.01, ***: p≤0.001, ****: p≤0.0001 by one-way ANOVA with multiple comparisons.

ELISA testing performed for the presence of the SARS-COV-2 receptor binding domain (RBD)-specific antibodies is shown in FIG. 17 panel D. The RBD domain is the part of trimeric Spike that recognizes the human ACE-2 receptor and mediates virus entry. Hence, this assay is a surrogate for protective immunity. This uses a commercial ELISA (Acro Biosystems RAS-T091). Shown is a box and whisker plot of range, median, 1st and 3rd quartile overlayed with individual data points for either S negative group (blue) or the S positive group (brown). The amount of anti-S specific IgG is shown on the vertical axis in ng/ml on a log 10 scale. Significance is indicated by the number of starts with *: p≤0.05, **: p≤0.01, ***: p≤0.001, **** p≤0.0001 by one-way ANOVA with multiple comparisons.

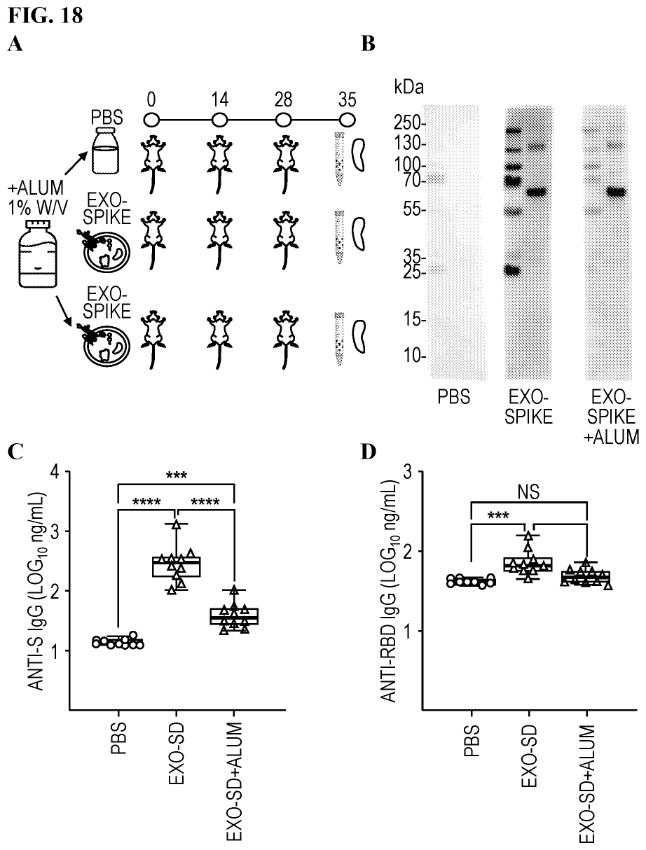

Further repeat experiments were performed as shown in FIG. 18. FIG. 18 panel A provides an illustration of the treatment plan and boost regimen. Numbers on top indicate days of injection or (day 35) collection. Alum at 1% w/v was added as a carrier in some groups. The group size was n=10. Western-Blot assays were performed to test sera for the presence of anti-SARS-COV-2 Spike antibodies (FIG. 18 panel B). The target Spike-His protein (construct pDD3503) was collected from conditioned cell media and purified using a His column. Protein was run on an SDS-PAGE gel. Serum from mice injected with PBS, Exo-Spike, or Exo-Spike alum was incubated with the membranes at 1:100 dilution and detected with anti-murine total IgG-conjugated to horseradish peroxidase. Dark bands indicate that serum antibodies from the mice recognize the full-length SARS-COV-2 spike protein and its break-down products. Weak bands on the left and numbers indicate molecular weight markers in kDa.

Results of an ELISA assay testing for the presence of trimer-Spike antibodies is shown in FIG. 18 panel C. Trimeric Spike, rather than monomeric Spike is the native species that recognizes the human ACE-2 receptor and mediates virus entry. Hence, this assay is a surrogate for protective immunity. This uses a commercial ELISA (Acro Biosystems RAS-T023). Shown is a box and whisker plot of range, median, 1st and 3rd quartile overlayed with individual data points for either S negative group (blue) or the S positive group (brown). The amount of anti-S specific IgG is shown on the vertical axis in ng/ml on a log 10 scale. Significance is indicated by the number of starts with *: p<0.05, **: p=0.01, ***: p<0.001, ****: p=0.0001 by one-way ANOVA with multiple comparisons. Results from ELISA testing for the presence of RBD-specific antibodies is shown in FIG. 18 panel D. ELISA testing for the presence of the SARS-CoV-2 receptor binding domain (RBD)-specific antibodies. The RBD domain is the part of trimeric Spike that recognizes the human ACE-2 receptor and mediates virus entry. Hence, this assay is a surrogate for protective immunity. This uses a commercial ELISA (Acro Biosystems RAS-T091). The amount of anti-S specific IgG is shown on the vertical axis in ng/ml on a log 10 scale. Significance is indicated by the number of starts with *: p≤0.05, **: p≤0.01, p≤0.001, ****: p≤0.0001 by one-way ANOVA with multiple comparisons.

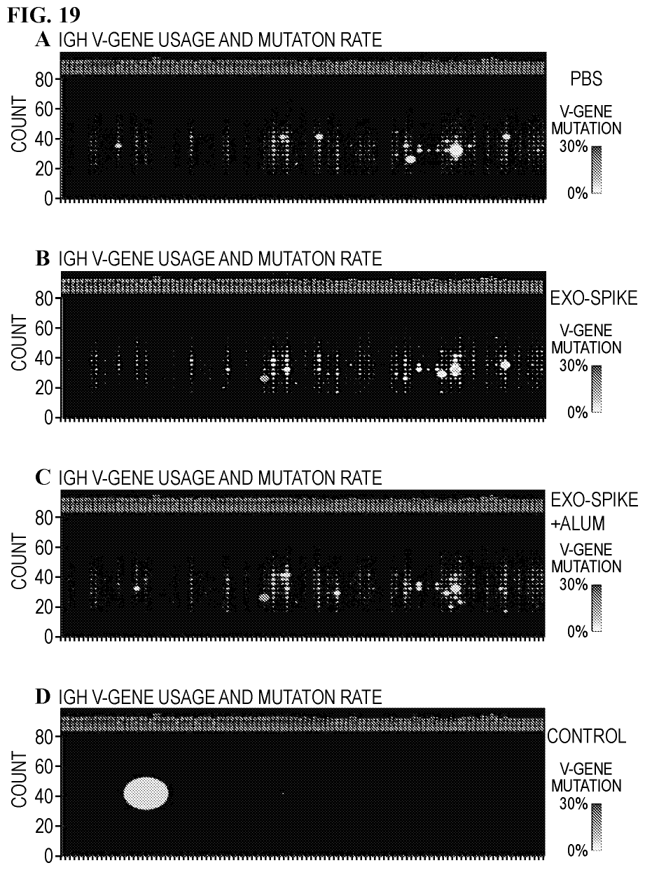

B-cell receptor sequencing of bone marrow was performed following the above experiments. These analyses showed altered V-gene mutations after “ExoSpike” vaccination. Another orthogonal measure of vaccine action is a change in the B cell receptor repertoire of the recipient mice. Here, bone marrow was harvested from the femurs of mice injected with (FIG. 19 panel A) PBS, (FIG. 19 panel B) Exo-Spike, (FIG. 19 panel C) Exo-Spike+Alum, control (FIG. 19 panel D) as described above. Red blood cells were lysed using a lysis buffer (Sigma R7757), then white blood cells were spun down and added to RNAlater (Invitrogen AM7022). Three mice from each treatment were grouped together. Then, the nucleic acid was amplified and barcoded using the Ion AmpliSeq (A45486). The DNA was read on the Ion Torrent sequencer, and data was analyzed using the ThermoFisher software to determine B-cell repertoire diversity. The purple dots in (FIG. 19 panel C) and (FIG. 19 panel D) show areas of high mutation rates.

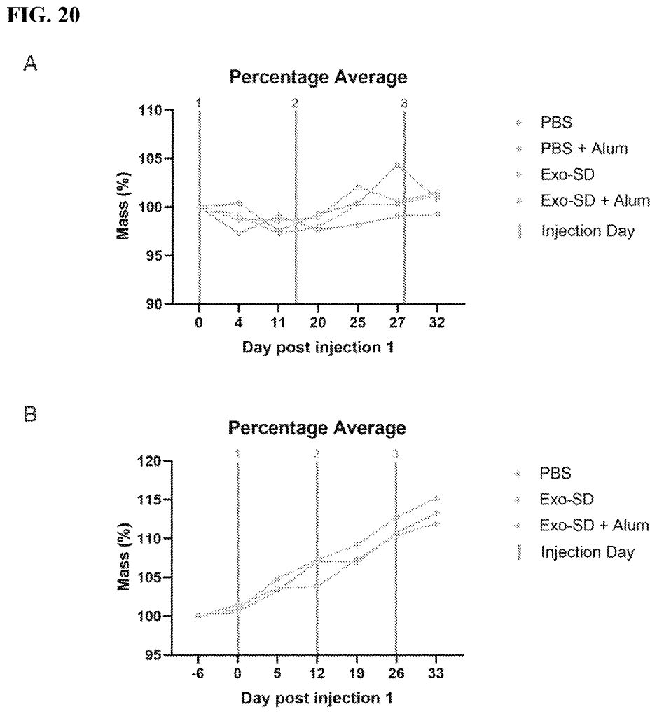

Experiments were also performed to evaluate the safety of “ExoSpike” formulation (FIG. 20). Injection with Exo-Spike did not cause any deleterious effects on the mice. The animal facilities core collected weight data for the mice during the different trials. Mice were weighed at the start of injections (FIG. 20 panel A) or 6 days before injections started (FIG. 2 panel B). All mice were weighed an additional two times. The average weights were then graphed using GraphPad. There were no significant differences between the weights of different treatment groups.

TABLE 1
Non-limiting examples of tetraspanin proteins.

| Protein | Gene | Aliases |
|---|---|---|
| TSPAN1 | TSPAN1 | TSP-1 |
| TSPAN2 | TSPAN2 | TSP-2 |
| TSPAN3 | TSPAN3 | TSP-3 |
| TSPAN4 | TSPAN4 | TSP-4, NAG-2 |
| TSPAN5 | TSPAN5 | TSP-5 |
| TSPAN6 | TSPAN6 | TSP-6 |
| TSPAN7 | TSPAN7 | CD231/TALLA-1/A15 |
| TSPAN8 | TSPAN8 | CO-029 |
| TSPAN9 | TSPAN9 | NET-5 |
| TSPAN10 | TSPAN10 | OCULOSPANIN |
| TSPAN11 | TSPAN11 | CD151-like |
| TSPAN12 | TSPAN12 | NET-2 |
| TSPAN13 | TSPAN13 | NET-6 |
| TSPAN14 | TSPAN14 | |
| TSPAN15 | TSPAN15 | NET-7 |
| TSPAN16 | TSPAN16 | TM4-B |
| TSPAN17 | TSPAN17 | |
| TSPAN18 | TSPAN18 | |
| TSPAN19 | TSPAN19 | |
| TSPAN20 | UPK1B | UP1b, UPK1B |
| TSPAN21 | TSPAN21 | UP1a, UPK1A |
| TSPAN22 | PRPH2 | RDS, PRPH2 |
| TSPAN23 | TSPAN23 | ROM1 |
| TSPAN24 | CD151 | CD151 |
| TSPAN25 | CD53 | CD53 |
| TSPAN26 | CD37 | CD37 |
| TSPAN27 | CD82 | CD82 |
| TSPAN28 | CD81 | CD81 |
| TSPAN29 | CD9 | CD9 |
| TSPAN30 | CD63 | CD63 |
| TSPAN31 | TSPAN31 | SAS |
| TSPAN32 | TSPAN32 | TSSC6 |
| TSPAN33 | TSPAN33 | |

The foregoing is illustrative of the present invention, and is not to be construed as limiting thereof. The invention is defined by the following claims, with equivalents of the claims to be included therein.

1. A synthetic tetraspanin protein comprising:
a tetraspanin backbone comprising an N-terminus, a C-terminus, a first, second, third and fourth transmembrane segment, a first extracellular loop between the first and second transmembrane segment, and a second extracellular loop between the third and fourth transmembrane segments; and
a heterologous protein or fragment thereof inserted within the second extracellular loop of the tetraspanin backbone, wherein the heterologous protein or fragment thereof is at least 100 amino acids in length.

2. The synthetic tetraspanin protein of claim 1, wherein the inserted heterologous protein or fragment thereof is about 500 to about 1500 amino acids in length.

3. (canceled)

4. The synthetic tetraspanin protein of claim 1, wherein the heterologous protein or fragment thereof expresses proper protein folding.

5. (canceled)

6. The synthetic tetraspanin of claim 1, wherein the heterologous protein or fragment thereof comprises a viral transmembrane protein or fragment thereof.

7. (canceled)

8. The synthetic tetraspanin of claim 1, wherein the heterologous protein or fragment thereof comprises a coronavirus Spike(S) protein and/or Spike-receptor binding protein (RBD), an HIV gp120 and/or gp41, any fragment and/or variant thereof, or any combination thereof.

9. (canceled)

10. The synthetic tetraspanin of claim 89, wherein the coronavirus S protein comprises or consists of an amino acid sequence at least 90% identical to any one of:
(a) SEQ ID NO:1 (Spike (Lineage B.1.2., MT565498.1)
(b) SEQ ID NO:2 (Delta Spike Variant); or
(c) SEQ ID NO:3 (Stabilized Delta Spike Variant).

11. The synthetic tetraspanin protein of claim 1, wherein the heterologous protein or fragment thereof is inserted into the tetraspanin backbone at a position within about amino acid position 114 to about amino acid position 199, wherein the numbering corresponds to the amino acid sequence of a reference tetraspanin identified as GenBank Acc. No. NP_004347.1 and/or CCDS 7734.1.

12. The synthetic tetraspanin protein of claim 10, wherein the heterologous protein or fragment thereof is inserted into the tetraspanin backbone immediately following amino acid position 150 (e.g., between amino acid position 150 and 151), wherein the numbering corresponds to the amino acid sequence of the reference tetraspanin.

13. The synthetic tetraspanin protein of claim 1, wherein the tetraspanin backbone comprises a backbone of any one of TABLE 1.

14. The synthetic tetraspanin protein of claim 1, wherein the tetraspanin backbone comprise the amino acid sequence of MGVEGCTKCIKYLLFVFNFVFWLAGGVILGVALWLRHDPQTTNLLYLELGDKPAPNTF YVGIYILIAVGAVMMFVGFLGCYGAIQESQCLLGTFFTCLVILFACEVAAGIWGFVNKD QIAKDVKQFYDQALQQAVVDDDANNAKAVVKTFHETLDCCGSSTLTALTTSVLKNNL CPSGSNIISNLFKEDCHQKIDDLFSGKLYLIGIAAIVVAVIMIFEMILSMVLCCGIRNSSVY (SEQ ID NO:6) or an amino acid sequence at least 90% identical thereto.

15. The synthetic tetraspanin protein of claim 1, further comprising a second insertion within the first extracellular loop.

16. (canceled)

17. The synthetic tetraspanin protein of claim 15, wherein the second insertion is inserted into the tetraspanin backbone at a position within about amino acid position 35 to about amino acid position 57 (e.g., between amino acid position 38 and 39, e.g., immediately following amino acid position 38), wherein the numbering corresponds to the amino acid sequence of a reference tetraspanin identified as GenBank Acc. No. NP_004347.1 and/or CCDS 7734.1.

18. (canceled)

19. The synthetic tetraspanin protein of claim 1, further comprising a moiety at the C-terminus.

20-21. (canceled)

22. An isolated nucleic acid molecule encoding the synthetic tetraspanin protein of claim 1.

23. A vector comprising and/or encoding the synthetic tetraspanin protein of claim 1, optionally wherein the vector is an extracellular vesicle.

24-27. (canceled)

28. A population of extracellular vesicles comprising the synthetic tetraspanin protein of claim 1.

29. A composition comprising the synthetic tetraspanin protein of claim 1, further comprising a pharmaceutically acceptable carrier, diluent, and/or adjuvant.

30. (canceled)

31. A method of producing an immune response to an infection in a subject, comprising administering to the subject an effective amount of the synthetic tetraspanin protein of claim 1.

32-35. (canceled)

36. A method of treating to an infection in a subject, comprising administering to the subject an effective amount of the synthetic tetraspanin protein of claim 1.

37. (canceled)

38. The method of claim 36, wherein the viral infection is a poxvirus, a herpesvirus, human immunodeficiency virus (HIV), and/or a coronavirus infection.

39-54. (canceled)
